(19) United States
(12) Patent Application Publication
(10) Pub. No.: US 2024/0076376 A1
(43) Pub. Date: Mar. 7, 2024

(54) APPROVED PRODUCTS FOR THE TREATMENT OF MULTIPLE MYELOMA

(71) Applicant: JANSSEN BIOTECH, INC., Horsham, PA (US)

(72) Inventors: Jenna Goldberg, Livingston, NJ (US); Arnob Banerjee, Baltimore, MD (US)

(21) Appl. No.: 18/452,751

(22) Filed: Aug. 21, 2023

Related U.S. Application Data
(60) Provisional application No. 63/400,228, filed on Aug. 23, 2022.
(60) Provisional application No. 63/400,527, filed on Aug. 24, 2022.
(60) Provisional application No. 63/419,105, filed on Oct. 25, 2022.
(60) Provisional application No. 63/426,911, filed on Nov. 21, 2022.
(60) Provisional application No. 63/514,953, filed on Jul. 21, 2023.

(51) Int. Cl. C07K 16/28; A61K 31/138; A61K 31/167; A61K 31/573; A61K 39/395; A61K 47/12; A61K 47/18; A61K 47/26; A61P 35/00

(52) U.S. Cl. CPC C07K 16/2809; A61K 31/138; A61K 31/167; A61K 31/573; A61K 39/3955; A61K 47/12; A61K 47/183; A61K 47/26; A61P 35/00; C07K 16/2878; A61K 2039/505; C07K 2317/24

(57) ABSTRACT

Described herein are approved products and methods of using approved products for treating relapsed or refractory multiple myeloma in a patient. Also described herein are methods of selling or offering for sale an approved product.

### CROSS-REFERENCE TO RELATED APPLICATIONS

This application claims priority to U.S. Provisional Application No. 63/400,228, which was filed on Aug. 23, 2022, U.S. Provisional Application No. 63/400,527, which was filed on Aug. 24, 2022, U.S. Provisional Application No. 63/419,105, which was filed on Oct. 25, 2022; U.S. Provisional Application No. 63/426,911, which was filed on Nov. 21, 2022, and U.S. Provisional Application No. 63/514,953, which was filed on Jul. 21, 2023, the contents of each of which are hereby incorporated by reference in their entirety.

### SEQUENCE LISTING

The instant application contains a Sequence Listing which is being submitted herewith electronically in XML format and is hereby incorporated by reference in its entirety. Said XML copy, created on Aug. 1, 2023, is named 103693005149_Sequence_Listing.xml and is 16,290 bytes in size.

### TECHNICAL FIELD

Disclosed herein are approved products and methods of using approved products to treat relapsed or refractory multiple myeloma (MM).

### BACKGROUND

In the pharmaceutical industry, the path from drug discovery to reaching an approved product is riddled with uncertainty. That path is one that since 2010 and out of more than 12,000 clinical studies, there is very low likelihood of regulatory approval of less than 10% (from Phase 1 to approval). The final path to approval is also not a certainty nor inevitable as failures continue as late as from Phase 3 to approval. This is not only due to the high unpredictability and uncertainty regarding how a pharmaceutical product will impact human biology, but often human behavior is unpredictable in how data is interpreted by regulatory agencies and whether the data warrants approving a study drug as an approved product for use in commerce.

As of February 2022, the five-year survival rate for people with multiple myeloma (MM) in the United States was 55%. Unfortunately, it is estimated that more than 12,000 people will die from the disease in 2022. cancer.net/cancer-types/multiple-myeloma/statistics.

Between the year 2000 and as of the filing of this document, there had been more than 200 molecules assessed in clinical trials with the hope of identifying a treatment for patients suffering from MM. (Pharmalntelligence, Pharmaprojects database results using terms “Cancer, myeloma”). Of those molecules, only nineteen (19) pioneer molecules had been incorporated into approved products and used to treat MM patients. Another 185 efforts to treat MM as either a lead molecule or in combination were suspended and did not progress. This data makes clear that the path to effective patient treatments for MM remains complex and unpredictable to this day. Despite the existence of the nineteen approved medicines, there remains a significant unmet patient need to treat patients with MM.

### SUMMARY

Provided herein are approved products comprising: 30 mg/3 mL (10 mg/mL) of teclistamab-cqyv in a single-dose vial; or 153 mg/1.7 mL (90 mg/mL) of teclistamab-cqyv in a single-dose vial.

Also provided herein are pharmaceutical products comprising teclistamab-cqyv in a single-dose vial for injection, wherein the pharmaceutical product is packaged, and wherein the package includes a label that identifies the pharmaceutical product as indicated as monotherapy for the treatment of adult patients with relapsed or refractory multiple myeloma, who have received at least four prior lines of therapy, including an immunomodulatory agent, a proteasome inhibitor, and an anti-CD38 monoclonal antibody.

Provided herein is a combination of a corticosteroid, a histamine-1 (H1) receptor antagonist, an antipyretic, and an approved product.

Kits comprising the disclosed approved products or pharmaceutical products are also provided.

Provided herein are methods for treating relapsed or refractory multiple myeloma in a patient, the methods comprising administering the disclosed approved products to the patient in an amount and manner that is described in a drug product label for the approved product.

Provided herein are methods for treating relapsed or refractory multiple myeloma in a patient, the methods comprising administering a corticosteroid, a H1 receptor antagonist, an antipyretic, and the disclosed approved products to the patient in an amount and manner that is described in a drug product label for the approved product.

Also provided are methods of selling an approved product, said methods comprising selling such approved product, wherein an approved product label for a reference product for such approved product includes instructions for treating adult patients with relapsed or refractory multiple myeloma after at least four prior lines of therapy, including a proteasome inhibitor, an immunomodulatory agent, and an anti-CD38 monoclonal antibody.

Provided herein are methods of offering for sale an approved product, said methods comprising offering for sale such approved product, wherein an approved product label for a reference product for such approved product includes instructions for treating adult patients with relapsed or refractory multiple myeloma after at least four prior lines of therapy, including a proteasome inhibitor, an immunomodulatory agent, and an anti-CD38 monoclonal antibody.

### DETAILED DESCRIPTION OF ILLUSTRATIVE EMBODIMENTS

The disclosed approved products and methods may be understood more readily by reference to the following detailed description. It is to be understood that the disclosed approved products and methods are not limited to the specific approved products and methods described and/or shown herein, and that the terminology used herein is for the purpose of describing particular embodiments by way of example only and is not intended to be limiting of the claimed approved products and methods.

Unless specifically stated otherwise, any description as to a possible mechanism or mode of action or reason for improvement is meant to be illustrative only, and the disclosed approved products and methods are not to be constrained by the correctness or incorrectness of any such suggested mechanism or mode of action or reason for improvement.

Throughout this text, the descriptions refer to approved products and methods. Where the disclosure describes or claims a feature or embodiment associated with an approved product, such a feature or embodiment is equally applicable to the methods. Likewise, where the disclosure describes or claims a feature or embodiment associated with the methods, such a feature or embodiment is equally applicable to the approved products.

When a value is expressed as an approximation by use of the descriptor “about,” it will be understood that the particular value forms another embodiment. Reference to a particular numerical value includes at least that particular value, unless the context clearly dictates otherwise. In general, use of the term “about” indicates approximations that can vary depending on the desired properties sought to be obtained by the disclosed subject matter and is to be interpreted in the specific context in which it is used, based on its function. In some cases, the number of significant figures used for a particular value may be one non-limiting method of determining the extent of the word “about.” In other cases, the gradations used in a series of values may be used to determine the intended range available to the term “about” for each value.

The term “about” as used herein when referring to a measurable value such as an amount, a temporal duration, and the like, is meant to encompass variations of up to ±10% from the specified value. Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

Where a range of numerical values is recited or established herein, the range includes the endpoints thereof and all the individual integers and fractions within the range, and also includes each of the narrower ranges therein formed by all the various possible combinations of those endpoints and internal integers and fractions to form subgroups of the larger group of values within the stated range to the same extent as if each of those narrower ranges was explicitly recited. Where a range of numerical values is stated herein as being greater than a stated value, the range is nevertheless finite and is bounded on its upper end by a value that is operable within the context of the herein disclosure. Where a range of numerical values is stated herein as being less than a stated value, the range is nevertheless bounded on its lower end by a non-zero value. It is not intended that the scope of the approved products and methods be limited to the specific values recited when defining a range. All ranges are inclusive and combinable.

When a list is presented, unless stated otherwise, it is to be understood that each individual element of that list, and every combination of that list, is a separate embodiment. For example, a list of embodiments presented as “A, B, or C” is to be interpreted as including the embodiments, “A,” “B,” “C,” “A or B,” “A or C,” “B or C,” or “A, B, or C.”

It is to be appreciated that certain features of the disclosed approved products and methods which are, for clarity, described herein in the context of separate embodiments, may also be provided in combination in a single embodiment. Conversely, various features of the disclosed approved products and methods that are, for brevity, described in the context of a single embodiment, may also be provided separately or in any subcombination.

As used herein, the singular forms “a,” “an,” and “the” include the plural.

Various terms relating to aspects of the description are used throughout the specification and claims. Such terms are to be given their ordinary meaning in the art unless otherwise indicated. Other specifically defined terms are to be construed in a manner consistent with the definitions provided herein.

The term “comprising” is intended to include examples encompassed by the terms “consisting essentially of” and “consisting of”; similarly, the term “consisting essentially of” is intended to include examples encompassed by the term “consisting of”

The term “approved product” means: a pharmaceutical product that i) has been approved for introduction into commerce by a regulatory agency for use in treating humans with relapsed or refractory multiple myeloma who have received at least four prior therapies, including a proteasome inhibitor, an immunomodulatory agent, and an anti-CD38 monoclonal antibody; ii) includes a biological product having at least the amino acid sequences of SEQ ID NOs: 1, 2, 3, and 4; iii) is formulated for delivery by injection in either a 10 mg/ml or 90 mg/ml concentration; and iv) induces an overall response rate (ORR) of at least 61.8% in humans who previously received at least three prior therapies, including a proteasome inhibitor, an immunomodulatory agent, and an anti-CD38 monoclonal antibody. An example of the “approved product” is the product listed in the approved product label of Example 1.

The term “biosimilar” means a product approved by a regulatory agency comprising a biological product that is highly similar to and does not cause any clinically meaningful differences when administered to patients relative to a pioneer company's reference product approved product. A biosimilar can be shown to be highly similar to the reference product by extensively analyzing (i.e., characterizing) the structure and function of both the reference product and the proposed biosimilar and comparing characteristics of the products, such as purity, chemical identity, and bioactivity. Note that different oligosaccharide profiles may exist between products produced using different cell lines or manufacturing techniques and that a biosimilar may behave differently in patients if manufactured using a different cell line or a different methodology.

The manufacturer of a proposed biosimilar product generates data comparing the proposed product to the approved reference product in order to demonstrate biosimilarity. A biosimilar product application must include data demonstrating biosimilarity to the reference product, which includes data from:
Analytical studies demonstrating that the biological product is highly similar to the reference product, notwithstanding minor differences in clinically inactive components;
Animal studies, including an assessment of toxicity; and
A clinical study or studies sufficient to demonstrate safety, purity, and potency of the proposed biosimilar product in one or more of the indications for which the reference product is licensed, which typically includes assessing immunogenicity, pharmacokinetics (PK), and, in some cases, pharmacodynamics (PD) and may also include a comparative clinical study.

A biosimilar manufacturer may not need to conduct as many clinical trials and can simply rely on the innovative research, development, and clinical work of the pioneer company to bring its biosimilar forward into commerce.

The term “reference product” refers to a product that is approved by a regulatory agency (e.g. the approved product) against which a proposed biosimilar product is compared. A reference product is approved based on, among other things, a full complement of safety and effectiveness data. A proposed biosimilar product is compared to, and evaluated against, a reference product to ensure that the product is highly similar and has no clinically meaningful differences.

The term “overall response rate” (ORR) means the proportion of subjects who achieved partial response (PR) or better (PR, very good partial response (VGPR), complete response (CR), or stringent complete response (sCR)), during or after study treatment but before the start of subsequent anti-myeloma therapy. ORR was assessed using the International Myeloma Working Group (IMWG) 2016 response criteria as the primary endpoint. ORR is described further in Moreau, A. L., et al., Teclistamab in Relapsed or Refractory Multiple Myeloma, N Engl J Med (2022); 387:495-505, incorporated by reference herein. The criteria for ORR as it relates to the approved product described herein are further detailed in Example 1.

“Label” or “approved product label” refers to information provided to a patient which provides relevant information regarding the approved product. Such information includes, without limitation, one or more of: the description of the approved product, clinical pharmacology, indications (uses for the approved product), contraindication (who should not take the approved product), warnings, precautions, adverse events (side effects), drug abuse and dependence, dosage and administration, use in pregnancy, use in nursing mothers, use in children and older patients, how the approved product is supplied, safety information for the patient, or any combination thereof. In some embodiments, the label or approved product label identifies teclistamab-cqyv and provides instructions for its use in a patient.

“Antibody” refers to all isotypes of immunoglobulins (IgG, IgA, IgE, IgM, IgD, and IgY) including various monomeric, polymeric, and chimeric forms, unless otherwise specified. Specifically encompassed by the term “antibody” are polyclonal antibodies, monoclonal antibodies (mAbs), and antibody-like polypeptides, such as chimeric antibodies and humanized antibodies.

“BCMA” refers to human B cell maturation antigen, also known as CD269 and TNFRSF17 (UniProt Q02223), which is a member of the tumor necrosis receptor superfamily that is preferentially expressed in differentiated plasma cells. The extracellular domain of human BCMA contains, according to UniProt, amino acids 1-54 (or 5-51).

“CD3” refers to the human CD3 protein multi-subunit complex. The CD3 protein multi-subunit complex is composed to six distinctive polypeptide chains. These include a CD3γ chain (SwissProt P09693), a CD3δ chain (SwissProt P04234), two CDR chains (SwissProt P07766), and one CD3ζ chain homodimer (SwissProt 20963), and which is associated with the T cell receptor α and β chain. The term “CD3” includes any CD3 variant, isoform and species homolog which is naturally expressed by cells (including T cells).

Teclistamab-cqyv is a humanized immunoglobulin G4 (IgG4) bispecific B-cell maturation antigen (BCMA)-directed cluster of differentiation (CD3) T-cell engager. The molecular weight of teclistamab-cqyv is approximately 146 kDa for the predominant GOF/GOF glycoform. Teclistamab-cqvv has an anti-BCMA heavy chain and light chain and an anti-CD3 heavy chain and light chain with two interchain disulfide bonds connecting the two arms. It is produced in Chinese Hamster Ovary (CHO) cells using recombinant DNA technology and is prepared by controlled reduction and oxidation of the anti-BCMA mAb and the anti-CD3 mAb resulting in an exchange of the Fab arms. The Fab arm exchange was facilitated by amino acid substitutions at positions F410L and R414K in the CH3 domain of the parental anti-CD3 mAb HC (according to sequential numbering of amino acids) to enable preferential refolding of the heterodimer. Teclistamab-cqvv utilizes an IgG4 PAA scaffold containing Pro mutations (S229P in the anti-BCMA mAb and S233P in the anti-CD3 mAb, according to sequential numbering of amino acids) that stabilize the hinge region and Ala/Ala mutations (F235A, L236A in the anti-BCMA mAb and F239A, L240A in anti-CD3 mAb, according to sequential numbering of amino acids) that suppress FcγR binding. The resulting teclistamab-cqyv bispecific antibody comprises Fc substitutions, with the BMCA-binding arm and CD3-binding arm each comprising Pro/Ala/Ala substitutions at amino acid positions 228/234/235, respectively (according to EU index numbering); and the CD3-binding arm also comprising F405L and R409K substitutions (according to EU index numbering).

As used herein, the term “patient” refers to a human. Subject and patient can be used interchangeably throughout this disclosure.

As used herein, the term “refractory” refers to disease which becomes non-responsive or progressive on therapy.

The phrase “pharmaceutically acceptable,” as used in connection with compositions described herein, refers to molecular entities and other ingredients of such compositions that are physiologically tolerable and do not typically produce untoward reactions when administered to a human. Preferably, the term “pharmaceutically acceptable” means approved by a regulatory agency of the Federal or a state government or listed in the U.S. Pharmacopeia or other generally recognized pharmacopeia for use in mammals, and more particularly in humans.

The term “placebo” as used herein means administration of a pharmaceutical composition that does not include teclistamab-cqyv.

“Treat,” “treatment,” and like terms include reducing the severity and/or frequency of symptoms, eliminating symptoms and/or the underlying cause of the symptoms, reducing the frequency or likelihood of symptoms and/or their underlying cause, and improving or remediating damage caused, directly or indirectly, by the relapsed or refractory multiple myeloma. “Treatment” can also mean delaying or slowing of disease progression and/or prolonging survival as compared to expected survival if a subject was not receiving the treatment. Treatment includes, for example, achieving desired clinical results, such as the efficacy results provided in Example 1.

### Approved Product

Disclosed herein are approved products comprising: 30 mg/3 mL (10 mg/mL) of teclistamab-cqyv in a single-dose vial; and/or 153 mg/1.7 mL (90 mg/mL) of teclistamab-cqyv in a single-dose vial. In some embodiments, the approved product comprises 30 mg/3 mL (10 mg/mL) of teclistamab-cqyv in a single-dose vial. In some embodiments, the approved product comprises 153 mg/1.7 mL (90 mg/mL) of teclistamab-cqyv in a single-dose vial. In some embodiments, the approved product comprises 30 mg/3 mL (10 mg/mL) of teclistamab-cqyv in a single-dose vial and 153 mg/1.7 mL (90 mg/mL) of teclistamab-cqyv in a single-dose vial.

In an embodiment, the approved product is administered in step-up doses of 0.06 mg/kg and 0.3 mg/kg followed by 1.5 mg/kg once weekly until disease progression or unacceptable toxicity. In an embodiment, pretreatment medications are administered prior to each dose of the approved product's step-up dosing schedule, which includes step-up dose 1, step-up dose 2, and the first treatment dose as described in Table 1.

In some embodiments, the first step dose of 0.06 mg/kg is administered using the 10 mg/mL of teclistamab-cqyv in a single-dose vial, the second step dose of 0.3 mg/kg is administered using the 10 mg/mL of teclistamab-cqyv in a single-dose vial, and each treatment dose of 1.5 mg/kg is administered using the 90 mg/mL of teclistamab-cqyv in a single-dose vial.

The approved product can be formulated for delivery to a human in a dosing schedule comprising:
0.06 mg/kg administered as a first step-up dose on Day 1;
0.3 mg/kg administered as a second step-up dose 2 to 7 days after the first step-up dose;
1.5 mg/kg administered as a first treatment dose 2 to 7 days after the second step-up dose; and
1.5 mg/kg administered as a subsequent treatment dose one week after the first treatment dose and once weekly thereafter.

An exemplary dosing schedule is provided below:

Dosing

| schedule | Day | Dose | |
|---|---|---|---|
| Step-up | Day 1 | Step-up dose 1 | 0.06 mg/kg |
| dosing | Day 4^{b} | Step-up dose 2 | 0.3 mg/kg |
| schedule^{a} | Day 7^{c} | First treatment dose | 1.5 mg/kg |
| Weekly | One week after first | Subsequent | 1.5 mg/kg once |
| dosing | treatment dose and | treatment doses | weekly |
| schedule^{a} | weekly thereafter | | |

^{a}See Table 2 for recommendations on restarting TECVAYLI ® after dose delays.
^{b}Step-up dose 2 may be given between 2 to 4 days after step-up dose 1 and may be given up to 7 days after step-up dose 1 to allow for resolution of adverse reactions.
^{c}First treatment dose may be given between 2 to 4 days after step-up dose 2 and may be given up to 7 days after step-up dose 2 to allow for resolution of adverse reactions.

The approved product is administered until disease progression or unacceptable toxicity. For example, TECVAYLI® is administered until disease progression or unacceptable toxicity.

An alternative dosing schedule permits a reduction in the frequency of treatment doses if the subject exhibits any response for a minimum of 6 months. The approved product can be formulated for delivery to a human according to a dosing schedule comprising:
0.06 mg/kg administered as a first step-up dose on Day 1;
0.3 mg/kg administered as a second step-up dose 2 to 7 days after the first step-up dose;
1.5 mg/kg administered as a first treatment dose 2 to 7 days after the second step-up dose;
1.5 mg/kg administered as a subsequent treatment dose one week after the first treatment dose and once weekly thereafter; and
if the subject has any response for a minimum of 6 months, 1.5 mg/kg administered as a subsequent treatment dose bi-weekly (every two weeks).
   The term “any response” refers to any one of stringent complete response (sCR), complete response (CR), very good partial response (VGPR), and partial response (PR) according to IMWG 2016 criteria. As used herein, “bi-weekly” means once every two weeks.

An exemplary alternative dosing schedule is provided below:

Dosing

| | | |
|---|---|---|
| schedule | Day | Dose |

All patients

| | | | |
|---|---|---|---|
| Step-up dosing | Day 1 | Step-up dose 1 | 0.06 mg/kg |
| schedule^{a} | Day 4^{b} | Step-up dose 2 | 0.3 mg/kg |
| | Day 7^{c} | First treatment | 1.5 mg/kg |
| | | dose | |
| Weekly dosing | One week after first | Subsequent | 1.5 mg/kg |
| schedule^{a} | treatment dose and | treatment doses | once |
| | weekly thereafter | | weekly |

Patients who have any response^{d} for a minimum of 6 months

| | |
|---|---|
| Biweekly (every | Consider reducing the dosing frequency to |
| two weeks) | 1.5 mg/kg every two weeks |
| dosing schedule | |

^{a}See Table 2 for recommendations on restarting TECVAYLI ® after dose delays.
^{b}Step-up dose 2 may be given between two to four days after Step-up dose 1 and may be given up to 7 days after step-up dose 1 to allow for resolution of adverse reactions.
^{c}First treatment dose may be given between two to four days after Step-up dose 2 and may be given up to 7 days after step-up dose 2 to allow for resolution of adverse reactions.
^{d}“Any response” refers to any one of stringent complete response (sCR), complete response (CR), very good partial response (VGPR), and partial response (PR) according to IMWG 2016 criteria

Another alternative dosing schedule permits a reduction in the frequency of treatment doses if the subject exhibits a complete response for a minimum of 6 months. The approved product can be formulated for delivery to a human according to a dosing schedule comprising:
0.06 mg/kg administered as a first step-up dose on Day 1;
0.3 mg/kg administered as a second step-up dose 2 to 7 days after the first step-up dose;
1.5 mg/kg administered as a first treatment dose 2 to 7 days after the second step-up dose;
1.5 mg/kg administered as a subsequent treatment dose one week after the first treatment dose and once weekly thereafter; and
if the subject has a complete response or better for a minimum of 6 months, 1.5 mg/kg administered as a subsequent treatment dose bi-weekly (every two weeks).

If a subject exhibits a complete response or better, the response may comprise a complete response or a stringent complete response, according to IMWG 2016 criteria.

The term “step-up dosing” refers to ascending concentrations of doses of the approved product administered prior to the weekly dosing schedule. The term “first step-up dose” refers to Step-up dose 1 in the above table. The term “second step-up dose” refers to Step-up dose 2 in the above table. The “subsequent treatment dose” can be administered one week after the first treatment dose and weekly thereafter. One or more subsequent treatment doses can be administered (e.g. once weekly on weeks 1, 2, 3, 4, 5, 6, 7, 8, or for more than 8 weeks after the first treatment dose). In an alternative dosing schedule, if the subject exhibits any response for a minimum of 6 months, the frequency of dosing can be reduced to 1.5 mg/kg every two weeks. In another alternative dosing schedule, if the subject exhibits a complete response or better for a minimum of 6 months, the frequency of dosing can be reduced to 1.5 mg/kg every two weeks.

In some embodiments, the second step-up dose is administered on Day 3, Day 4, or Day 5. In some embodiments, the second step-up dose is administered on Day 4.

In some embodiments, the first treatment dose is administered 2 to 4 days after the second step-up dose. In some embodiments, the first treatment dose is administered on Day 7.

The approved product can be formulated for delivery to a human in a dosing schedule comprising:
0.06 mg/kg administered as a first step-up dose on Day 1;
0.3 mg/kg administered as a second step-up dose on Day 4;
1.5 mg/kg administered as a first treatment dose on Day 7; and
1.5 mg/kg administered as a subsequent treatment dose one week after the first treatment dose and once weekly thereafter.

In some embodiments, the approved product is a biosimilar of teclistamab-cqyv.

The approved product can include the following excipients: edetate disodium; glacial acetic acid; polysorbate 20; sodium acetate; sucrose; and water for injection, USP.

The approved product can be in a 3 ml single-dose vial comprising 30 mg of teclistamab-cqyv, 0.054 mg edetate disodium, 0.72 mg glacial acetic acid, 1.2 mg polysorbate 20, 2.7 mg sodium acetate, 240 mg sucrose, and water for injection, USP.

The approved product can be in a 1.7 ml single-dose vial comprising 153 mg of teclistamab-cqyv, 0.031 mg edetate disodium, 0.41 mg glacial acetic acid, 0.68 mg polysorbate 20, 1.5 mg sodium acetate, 140 mg sucrose, and water for injection, USP.

In certain embodiments, the approved product can be formulated into any of the stable aqueous pharmaceutical compositions described in PCT Application No. PCT/US2022/079535, filed Nov. 9, 2022, which is incorporated by reference herein.

The approved products can be administered to the patient in combination with a corticosteroid, a H1 receptor antagonist, and antipyretics. Provided herein is a combination of a corticosteroid, a H1 receptor antagonist, an antipyretics, and an approved product. The corticosteroid, the H1 receptor antagonist, and the antipyretics can be administered prior to the first step-up dose, prior to the second step-up dose, prior to the first treatment dose, and/or prior to each subsequent treatment dose. In some embodiments, the combination can comprise a corticosteroid comprising oral or intravenous dexamethasone, a H1 receptor antagonist comprising oral or intravenous diphenhydramine, and an antipyretic comprising oral or intravenous acetaminophen. In some embodiments, the combination comprises 16 mg of oral or intravenous dexamethasone, 50 mg or equivalent of oral or intravenous diphenhydramine, and 650 mg to 1000 mg, or equivalent, oral or intravenous acetaminophen.

Following administration of the approved product, the median follow-up can be 7.4 months or 9 months. For example, with a median follow-up of 7.4 months among responders, the estimated duration of response (DOR) rate can be 90.6% (95% CI: 80.3%, 95.7%) at 6 months and 66.5% (95% CI: 38.8%, 83.9%) at 9 months.

The approved product comprises a first heavy chain (HC) (SEQ ID NO: 1) and a first light chain (LC) (SEQ ID NO: 2) that pair to form a first antigen-binding site that immunospecifically binds BCMA, and a second heavy chain (HC) (SEQ ID NO: 3) and a second light chain (LC) (SEQ ID NO: 4) that pair to form a second antigen-binding site that immunospecifically binds CD3. A biosimilar can comprise, for example, a first HC having 95%, 96%, 97%, 98%, 99%, or 100% sequence identity to SEQ ID NO: 1, a first LC having 95%, 96%, 97%, 98%, 99%, or 100% sequence identity to SEQ ID NO: 2, a second HC having 95%, 96%, 97%, 98%, 99%, or 100% sequence identity to SEQ ID NO: 3, and a second LC having 95%, 96%, 97%, 98%, 99%, or 100% sequence identity to SEQ ID NO: 4. The antigen-binding site that binds BCMA comprises a heavy chain complementarity determining region 1 (HCDR1) comprising the amino acid sequence of SEQ ID NO: 5, HCDR2 comprising the amino acid sequence of SEQ ID NO: 6, and HCDR3 comprising the amino acid sequence of SEQ ID NO: 7, and light chain CDR1 (LCDR1) comprising the amino acid sequence of SEQ ID NO: 8, LCDR2 comprising the amino acid sequence of SEQ ID NO: 9, and LCDR3 comprising the amino acid sequence of SEQ ID NO: 10. The antigen-binding site that binds CD3 comprises HCDR1 comprising the amino acid sequence of SEQ ID NO: 11, HCDR2 comprising the amino acid sequence of SEQ ID NO: 12, and HCDR3 comprising the amino acid sequence of SEQ ID NO: 13, and LCDR1 comprising the amino acid sequence of SEQ ID NO: 14, LCDR2 comprising the amino acid sequence of SEQ ID NO: 15, and LCDR3 comprising the amino acid sequence of SEQ ID NO: 16. Antibody sequences are provided in the below table:

| Name | SEQUENCE |
|---|---|
| BCMA HC | QLQLQESGPGLVKPSETLSLTCTVSGGSISSGSY |
| (SEQ ID NO: 1) | FWGWIRQPPGKGLEWIGSIYYSGITYYNPSLKSR |
| | VTISVDTSKNQFSLKLSSVTAADTAVYYCARHDG |
| | AVAGLFDYWGQGTLVTVSSASTKGPSVFPLAPCS |
| | RSTSESTAALGCLVKDYFPEPVTVSWNSGALTSG |
| | VHTFPAVLQSSGLYSLSSVVTVPSSSLGTKTYTC |
| | NVDHKPSNTKVDKRVESKYGPPCPPCPAPEAAGG |
| | PSVFLFPPKPKDTLMISRTPEVTCVVVDVSQEDP |
| | EVQFNWYVDGVEVHNAKTKPREEQFNSTYRVVSV |
| | LTVLHQDWLNGKEYKCKVSNKGLPSSIEKTISKA |
| | KGQPREPQVYTLPPSQEEMTKNQVSLTCLVKGFY |
| | PSDIAVEWESNGQPENNYKTTPPVLDSDGSFFLY |
| | SRLTVDKSRWQEGNVFSCSVMHEALHNHYTQKSL |
| | SLSLGK |
| BCMA LC | SYVLTQPPSVSVAPGQTARITCGGNNIGSKSVHW |
| (SEQ ID NO: 2) | YQQPPGQAPVVVVYDDSDRPSGIPERFSGSNSGN |
| | TATLTISRVEAGDEAVYYCQVWDSSSDHVVFGGG |
| | TKLTVLGQPKAAPSVTLFPPSSEELQANKATLVC |
| | LISDFYPGAVTVAWKGDSSPVKAGVETTTPSKQS |
| | NNKYAASSYLSLTPEQWKSHRSYSCQVTHEGSTV |
| | EKTVAPTECS |
| CD3 HC | EVQLVESGGGLVQPGGSLRLSCAASGFTFNTYAM |
| (SEQ ID NO: 3) | NWVRQAPGKGLEWVARIRSKYNNYATYYAASVKG |
| | RFTISRDDSKNSLYLQMNSLKTEDTAVYYCARHG |
| | NFGNSYVSWFAYWGQGTLVTVSSASTKGPSVFPL |
| | APCSRSTSESTAALGCLVKDYFPEPVTVSWNSGA |
| | LTSGVHTFPAVLQSSGLYSLSSVVTVPSSSLGTK |
| | TYTCNVDHKPSNTKVDKRVESKYGPPCPPCPAPE |
| | AAGGPSVFLFPPKPKDTLMISRTPEVTCVVVDVS |
| | QEDPEVQFNWYVDGVEVHNAKTKPREEQFNSTYR |
| | VVSVLTVLHQDWLNGKEYKCKVSNKGLPSSIEKT |
| | ISKAKGQPREPQVYTLPPSQEEMTKNQVSLTCLV |
| | KGFYPSDIAVEWESNGQPENNYKTTPPVLDSDGS |
| | FLLYSKLTVDKSRWQEGNVFSCSVMHEALHNHYT |
| | QKSLSLSLGK |
| CD3 LC | QTVVTQEPSLTVSPGGTVTLTCRSSTGAVTTSNY |
| (SEQ ID NO: 4) | ANWVQQKPGQAPRGLIGGTNKRAPGTPARFSGSL |
| | LGGKAALTLSGVQPEDEAEYYCALWYSNLWVFGG |
| | GTKLTVLGQPKAAPSVTLFPPSSEELQANKATLV |
| | CLISDFYPGAVTVAWKADSSPVKAGVETTTPSKQ |
| | SNNKYAASSYLSLTPEQWKSHRSYSCQVTHEGST |
| | VEKTVAPTECS |
| BCMA HCDR1 | GGSISSGSYF |
| (SEQ ID NO: 5) | |
| BCMA HCDR2 | SIYYSGITYYNPSLKS |
| (SEQ ID NO: 6) | |
| BCMA HCDR3 | HDGAVAGLFDY |
| (SEQ ID NO: 7) | |
| BCMA LCDR1 | GGNNIGSKSVH |
| (SEQ ID NO: 8) | |
| BCMA LCDR2 | DDSDRPS |
| (SEQ ID NO: 9) | |
| BCMA LCDR3 | QVWDSSSDHVV |
| (SEQ ID NO: 10) | |
| CD3 HCDR1 | GFTFNTYAMN |
| (SEQ ID NO: 11) | |
| CD3 HCDR2 | RIRSKYNNYATYY |
| (SEQ ID NO: 12) | |
| CD3 HCDR3 | HGNFGNSYVSWFAY |
| (SEQ ID NO: 13) | |
| CD3 LCDR1 | RSSTGAVTTSNYAN |
| (SEQ ID NO: 14) | |
| CD3 LCDR2 | GTNKRAP |
| (SEQ ID NO: 15) | |
| CD3 LCDR3 | ALWYSNLWV |
| (SEQ ID NO: 16) | |

Also provided herein are pharmaceutical products comprising teclistamab-cqyv in a single-dose vial, wherein the pharmaceutical product is packaged, and wherein the package includes a label that identifies the pharmaceutical product as indicated as monotherapy for the treatment of adult patients with relapsed or refractory multiple myeloma, who have received at least four prior lines of therapies, including an immunomodulatory agent, a proteasome inhibitor, and an anti-CD38 monoclonal antibody.

Also provided herein is an approved drug product with at least one approved indication, wherein said approved drug product comprises teclistamab-cqyv.

Also provided herein is a drug product comprising teclistamab-cqyv, wherein the drug product is approved for treating multiple myeloma.

In an embodiment, an approved drug product comprises teclistamab-cqyv and such approved drug product improves anti-cancer cell activity as measured by overall response rate, duration of response (DOR), DOR rate, or time to first response, relative to a patient with multiple myeloma who is not receiving treatment with a bispecific antibody.

In an embodiment, an approved drug product comprises teclistamab-cqyv and is reference listed on the basis of data demonstrating an improvement in anti-cancer cell activity as measured by overall response rate, duration of response (DOR), DOR rate, or time to first response, relative to a patient with multiple myeloma who is not receiving treatment with a bispecific antibody.

Methods of Treatment with Approved Product

Disclosed herein are methods for treating relapsed or refractory multiple myeloma in a patient, the method comprising administering the disclosed approved products to the patient in an amount and manner that is described in a drug product label for the approved product. In some embodiments, the approved product is teclistamab-cqyv.

It is to be understood that the method of treatment embodiments described herein encompass the use of an approved product for the manufacture of a medicament and the approved product for use in treatment. Thus, also described herein are uses of the approved product in the manufacture of a medicament for the treatment of multiple myeloma in a patient, wherein the medicament is administered in an amount that is described in the approved product label for said approved product.

Prior to the administering of the approved product, the methods comprise administering a corticosteroid, a H1 receptor antagonist, and antipyretics to the patient. In some embodiments, the corticosteroid is dexamethasone, the H1 receptor antagonist is diphenhydramine, and the antipyretic is acetaminophen. Thus, provided herein are methods for treating relapsed or refractory multiple myeloma in a patient, the methods comprising administering a corticosteroid, an antihistamine, antipyretics, and the disclosed approved products to the patient in an amount and manner that is described in a drug product label for the approved product. The corticosteroid can comprise oral or intravenous dexamethasone, the H1 receptor antagonist can comprise oral or intravenous diphenhydramine, and the antipyretic can comprise oral or intravenous acetaminophen. In some embodiments, the methods comprise administering 16 mg of oral or intravenous dexamethasone, 50 mg or equivalent of oral or intravenous diphenhydramine, 650 mg to 1000 mg, or equivalent, of oral or intravenous acetaminophen, and the disclosed approved products to the patient in an amount and manner that is described in a drug product label for the approved product. In some embodiments, the corticosteroid, the H1 receptor antagonist, and the antipyretics are administered to the patient prior to a step-up dose or a treatment dose.

In some embodiments, the patient has not had a stroke, has not had a seizure, has not had an allogenic stem cell transplantation within 6 months of receiving the approved product, does not have an Eastern Cooperative Oncology Group (ECOG) performance score of 2 or higher, has no known active CNS involvement or clinical signs of meningeal involvement of multiple myeloma, and has no active or documented history of autoimmune disease, with the exception of vitiligo, Type 1 diabetes, and prior autoimmune thyroiditis.

The administration of the approved product can provide improved anti-cancer cell activity as measured by overall response rate, duration of response (DOR), DOR rate, or time to first response, relative to a patient with multiple myeloma who is not receiving treatment with a bispecific antibody.

The administration of the approved product can provide improved anti-cancer cell activity as measured by overall response rate relative to a patient with multiple myeloma who is not receiving treatment with a bispecific antibody.

The administration of the approved product can provide improved anti-cancer cell activity as measured by complete response (CR) or better, including stringent complete response (sCR) and CR, data relative to a patient with multiple myeloma who is not receiving treatment with a bispecific antibody.

The administration of the approved product can provide improved anti-cancer cell activity as measured by very good partial response data relative to a patient with multiple myeloma who is not receiving treatment with a bispecific antibody.

The administration of the approved product can provide improved anti-cancer cell activity as measured by partial response data relative to a patient with multiple myeloma who is not receiving treatment with a bispecific antibody.

The administration of the approved product can provide improved anti-cancer cell activity as measured by stringent complete response data, complete response data, very good partial response data, and partial response data relative to a patient with multiple myeloma who is not receiving treatment with a bispecific antibody.

The administration of the approved product can provide improved anti-cancer cell activity as measured by duration of response relative to a patient with multiple myeloma who is not receiving treatment with a bispecific antibody.

The administration of the approved product can provide improved anti-cancer cell activity as measured by time to first response relative to a patient with multiple myeloma who is not receiving treatment with a bispecific antibody.

The administration of the approved product can provide improved anti-cancer cell activity as measured by duration of response rate relative to a patient with multiple myeloma who is not receiving treatment with a bispecific antibody.

The administration of the approved product can provide improved anti-cancer cell activity in a patient population as measured by overall response rate relative to a patient population with multiple myeloma who are not receiving treatment with a bispecific antibody.

The administration of the approved product can provide improved anti-cancer cell activity in a patient population as measured by complete response (CR) or better, including stringent complete response (sCR) and CR, data relative to a patient population with multiple myeloma who are not receiving treatment with a bispecific antibody.

The administration of the approved product can provide improved anti-cancer cell activity in a patient population as measured by very good partial response data relative to a patient population with multiple myeloma who are not receiving treatment with a bispecific antibody.

The administration of the approved product can provide improved anti-cancer cell activity in a patient population as measured by partial response data relative to a patient population with multiple myeloma who are not receiving treatment with a bispecific antibody.

The administration of the approved product can provide improved anti-cancer cell activity in a patient population as measured by stringent complete response data, complete response data, very good partial response data, and partial response data relative to a patient population with multiple myeloma who are not receiving treatment with a bispecific antibody.

The administration of the approved product can provide improved anti-cancer cell activity in a patient population as measured by duration of response relative to a patient population with multiple myeloma who are not receiving treatment with a bispecific antibody.

The administration of the approved product can provide improved anti-cancer cell activity in a patient population as measured by time to first response relative to a patient population with multiple myeloma who are not receiving treatment with a bispecific antibody.

The administration of the approved product can provide improved anti-cancer cell activity in a patient population as measured by duration of response rate relative to a patient population with multiple myeloma who are not receiving treatment with a bispecific antibody.

The improvement in anti-cancer cell activity can be relative to treatment with placebo.

The improvement in anti-cancer cell activity can be relative to no treatment.

The improvement in anti-cancer cell activity can be relative to standard of care.

The administration of the approved product can result in no more than a grade 2 adverse event.

The administration of an amount of the approved product can result in no more than a grade 3 adverse event.

The administration of an amount of the approved product can result in no more than a grade 4 adverse event.

Methods of Restarting Therapy after a Dose Delay

Provided herein are methods of restarting a subject's dosing regimen for a BCMAxCD3 bispecific antibody after the subject has initiated a planned dosing regimen for treatment of multiple myeloma, and a delay has occurred between doses of the BCMAxCD3 bispecific antibody during the planned dosing regimen. According to certain embodiments, a planned dosing regimen comprises a step-up dosing schedule that includes a first step-up dose of 0.06 mg/kg, a second step-up dose of 0.3 mg/kg between 2 to 7 days after the first step-up dose, a first treatment dose of 1.5 mg/kg between 2 to 7 days after the second step-up dose; and subsequent treatment doses of 1.5 mg/kg one week after the first treatment dose and weekly thereafter. According to certain embodiments, a planned dosing regimen comprises a step-up dosing schedule that includes a first step-up dose of 0.06 mg/kg, a second step-up dose of 0.3 mg/kg between 2 to 7 days after the first step-up dose, a first treatment dose of 1.5 mg/kg between 2 to 7 days after the second step-up dose; subsequent treatment doses of 1.5 mg/kg one week after the first treatment dose and weekly thereafter; and if the subject has any response for a minimum of 6 months, 1.5 mg/kg administered as a subsequent treatment dose bi-weekly (every two weeks). According to certain embodiments, a planned dosing regimen comprises a step-up dosing schedule that includes a first step-up dose of 0.06 mg/kg, a second step-up dose of 0.3 mg/kg between 2 to 7 days after the first step-up dose, a first treatment dose of 1.5 mg/kg between 2 to 7 days after the second step-up dose; subsequent treatment doses of 1.5 mg/kg one week after the first treatment dose and weekly thereafter; and if the subject has a complete response or better for a minimum of 6 months, 1.5 mg/kg administered as a subsequent treatment dose bi-weekly (every two weeks).

Dosing regimens of a pharmaceutical product are developed based on extensive pre-clinical and clinical data. Once a safe and effective dosing regimen is established for a given therapy (also referred to herein as a planned dosing regimen), subjects must adhere to the regimen or otherwise face significant risks, such as increased side effects and/or diminished efficacy, etc. However, it is not uncommon for subjects to experience dose delays, especially a subject with multiple myeloma that faces day-to-day health and lifestyle challenges. When subjects experience a delay between doses of a BCMAxCD3 bispecific antibody that is longer than the interval between those doses in the established (planned) dosing regimen, there is a need for methods of restarting the therapy that are safe and effective. The inventors have developed methods of restarting therapy on a BCMAxCD3 bispecific antibody that are tailored to the length and timing of a particular dose delay, i.e., a method is selected from several options based on the last dose amount administered to the subject and the number of days since the last dose administered. The methods enable subjects to receive a dosing regimen that is safe and effective in treating multiple myeloma, despite experiencing a delay in dosing.

According to certain embodiments, if a dose of the approved product is delayed, the patient restarts therapy based on Table A and resumes the weekly or bi-weekly (every 2 weeks) dosing schedule accordingly.

TABLE A
Recommendations for Restarting TECVAYLI ® after Dose Delay

| | Duration of | |
|---|---|---|
| | delay from | |
| Last dose | the last dose | |
| administered | administered | Action |
| Step-up dose 1 | More than | Restart TECVAYLI ® step-up dosing |
| | 7 days | schedule at step-up dose 1 (0.06 mg/kg).^{a} |
| Step-up dose 2 | 8 days to | Repeat step-up dose 2 (0.3 mg/kg)^{a} |
| | 28 days | and continue TECVAYLI ® step-up |
| | | dosing schedule. |
| | More than | Restart TECVAYLI ® step-up dosing |
| | 28 days^{b} | schedule at step-up dose 1 (0.06 mg/kg).^{a} |
| Any treatment | 8 days to | Continue TECVAYLI ® at last |
| dose | 28 days | treatment dose and schedule (see |
| | | Table 1). |
| | More than | Restart TECVAYLI ® step-up dosing |
| | 28 days^{b} | schedule at step-up dose 1 (0.06 mg/kg).^{a} |

^{a}Administer pretreatment medications prior to TECVAYLI ® dose and monitor patients accordingly.
^{b}Consider benefit-risk of restarting TECVAYLI ® in patients who require a dose delay of more than 28 days due to an adverse reaction.

### Pharmaceutical Compositions and Routes of Administration

In view of its useful pharmacological properties, the approved product described herein may be formulated into various pharmaceutical forms for administration purposes.

The pharmaceutical composition (e.g., formulation) can comprise the approved product and one or more pharmaceutically acceptable carriers, adjuvants, excipients, diluents, fillers, buffers, stabilizers, preservatives, lubricants, or other materials well known to those skilled in the art and optionally other therapeutic or prophylactic agents. In some embodiments, the approved product is teclistamab-cqyv.

To prepare the pharmaceutical compositions, an effective amount of the approved product is combined in intimate admixture with a pharmaceutically acceptable carrier, which carrier may take a wide variety of forms depending on the form of preparation desired for administration. The pharmaceutical compositions can comprise an effective amount of the approved product combined in intimate admixture with one or more pharmaceutically acceptable excipients. Suitable pharmaceutically acceptable excipients include edetate disodium, glacial acetic acid, polysorbate 20, sodium acetate, sucrose, and water for injection, USP.

The pharmaceutical compositions can be in any form suitable for oral, intravenous, intraperitoneal, subcutaneous, pulmonary, transdermal, intramuscular, intranasal, buccal, sublingual, or suppository administration. The pharmaceutical compositions preferably are suitable for parenteral administration. The term “parenteral,” as used herein, includes intravenous, intramuscular, subcutaneous, rectal, vaginal, and intraperitoneal administration. More preferably, the pharmaceutical compositions are suitable for peripheral systemic delivery by intravenous, intraperitoneal, or subcutaneous injection. Most preferably, the pharmaceutical composition of the approved product is administered by subcutaneous injection.

These pharmaceutical compositions are desirably in unitary dosage form suitable, preferably, for parenteral injection. For example, the pharmaceutical compositions can be prepared by mixing the biological product included in the approved product with optional pharmaceutically acceptable carriers, excipients or stabilizers (Remington's Pharmaceutical Sciences 16th edition, Osol, A. Ed. (1980)), in the form of lyophilized formulations or aqueous solutions. The pharmaceutical composition comprising the approved product can comprise one or more excipients selected from edetate disodium, glacial acetic acid, Polysorbate 20, sodium acetate, sucrose, and water.

Also provided herein are methods of preparing a pharmaceutical composition that comprises the approved product, in the form of an aqueous solution, characterized by combining the approved product with one or more excipients selected from the group consisting of edetate disodium, glacial acetic acid, Polysorbate 20, sodium acetate, sucrose, and water for injection, USP. The aqueous solution can comprise teclistamab-cqyv, edetate disodium, glacial acetic acid, Polysorbate 20, sodium acetate, sucrose, and water for injection, USP.

In some embodiments, the approved product is formulated to provide patients with a dose of about 0.06 mg/kg, 0.3 mg/kg, or 1.5 mg/kg of teclistamab-cqyv in accordance with the approved product label.

### Methods of Dosing and Treatment Regimens

The approved product is administered in an amount sufficient to exert its anti-cancer cell activity. The recommended dosing amounts, regimens, and other key elements of uses of the approved product is provided in Table 1 and Example 1. In general, a patient is provided with a step-up dose 1 of the approved product in the amount of 0.06 mg/kg on day 1, followed by a step-up dose 2 of the approved product in the amount of 0.3 mg/kg on day 4, followed by a first treatment dose of the approved product in the amount of 1.5 mg/kg on day 7. The patient is then provided with a weekly dosing schedule of the approved product in the amount of 1.5 mg/kg that begins one week after the first treatment dose and is continued weekly thereafter. There are other variations on dosing that are also described in Example 1. In some embodiments, the approved product is teclistamab-cqyv.

The patient is a patient who has received at least four prior lines of therapy for the treatment of multiple myeloma including a proteasome inhibitor, an immunomodulatory agent, and an anti-CD38 monoclonal antibody.

The patient is pre-treated with one or more secondary medicinal products prior to receiving the approved product. A patient may be administered one or more pre-treatment medicinal products about 10 minutes to 6 hours, more specifically 30 minutes to 4.5 hours, or more specifically 1 hour to 3 hours prior to administration of the approved product. The one or more pre-treatment medicinal products include a corticosteroid, H1 receptor antagonist, an antipyretic, and/or an anti-viral prophylaxis. In an embodiment, the corticosteroid is oral or intravenous dexamethasone. In an embodiment, the H1 receptor antagonist is oral or intravenous diphenhydramine. In an embodiment, the antipyretic is oral or intravenous acetaminophen. In an embodiment, the patient is administered about 16 mg of oral or intravenous dexamethasone, about 50 mg or equivalent of oral or intravenous diphenhydramine, and about 650 mg to 1000 mg, or equivalent, of oral or intravenous acetaminophen one to three hours prior to administration of a step-up dose or a treatment dose of the approved product. Additional details regarding pre-treatment are provided in Example 1.

Also provided herein are methods of reducing the risk of an adverse event in a patient that receives an approved product. In an embodiment, the method comprises reducing the risk of cytokine release syndrome (CRS) and/or neurologic toxicity, such as Immune Effector Cell-Associated Neurotoxicity Syndrome (ICANS), in a patient that receives the approved product. In an embodiment, the method comprises administering the approved product in accordance with a Risk Evaluation and Mitigation Strategy (REMS) program (e.g., as described at www TECVAYLIREMS, the contents of which are incorporated by reference herein). As used herein, a REMS program is a program required by a health authority to manage known or potential serious risks associated with an approved product to ensure the benefits of the approved product outweigh its risks. According to an embodiment, a method for treating relapsed or refractory multiple myeloma in a patient comprises administering the approved product in accordance with a REMS program.

According to an embodiment, the REMS program includes distributing educational materials to healthcare providers (HCPs) and pharmacies that describe risks of CRS and/or neurologic toxicity associated with the approved product. The REMS program may include Elements to Assure Safe Use (ETASU), which comprises requiring prescribers to complete a training program on the risks of CRS and/or neurologic toxicity associated with the approved product, in order to become certified to prescribe the approved product. According to an embodiment, the REMS program comprises requiring prescribers to counsel patients on how to recognize and respond to signs and symptoms of CDS and neurologic toxicity (e.g., ICANS); for example, the REMS program may include training prescribers to instruct a patient that the patient should be hospitalized for 48 hours after administration of all doses within the step-up dosing schedule for the approved product. According to an embodiment, the REMS program comprises requiring pharmacies and healthcare settings to become certified to dispense the approved product by completing a training program.

According to an embodiment, administering an approved product to a patient in accordance with a REMS program comprises, prior to administering the approved product to the patient: (1) becoming certified to prescribe the approved product by completing a training program about risks of CRS and/or neurologic toxicity associated with the approved product; and (2) counseling the patient on how to recognize and respond to signs and symptoms of CRS and neurologic toxicity (e.g., ICANS).

### Approved Products as Kits or Articles of Manufacture

Also provided herein are kits and articles of manufacture comprising the disclosed approved products or the disclosed pharmaceutical products (“kits” shall be construed to include the elements of the approved product as defined herein). Such kits include a package or container that is compartmentalized to receive one or more dosages of the approved product disclosed herein. Suitable containers include, for example, bottles or vials. The containers can be formed from a variety of materials such as glass or plastic.

In some embodiments, the approved product is teclistamab-cqyv.

The articles of manufacture provided herein contain packaging materials. Examples of pharmaceutical packaging materials include, but are not limited to, blister packs, bottles, tubes, bags, containers, syringes, and any packaging material suitable for a selected formulation and intended mode of administration and treatment.

A kit typically includes labels listing contents and/or instructions for use, and package inserts with instructions for use. A set of instructions will also typically be included.

A label is on or associated with the container. In an embodiment, a label is on a container when letters, numbers or other characters forming the label are attached, molded or etched into the container itself; a label is associated with a container when it is present within a receptacle or carrier that also holds the container, e.g., as a package insert. An example of a kit of the invention is a kit comprising TECVAYLI®. A further example is a kit comprising a biosimilar. An example of an article of manufacture of the invention is an article of manufacture comprising TECVAYLI®. A further example is an article of manufacture comprising a biosimilar.

A label is used to indicate that the contents of a kit or of an article of manufacture are to be used for a specific therapeutic application. The label also indicates directions for use of the contents, such as in the methods described herein. The kit or the article of manufacture can comprise the approved product label.

The kits may comprise one or more suitably aliquoted compositions or reagents to generate compositions of the disclosure. The components of the kits may be packaged either in aqueous media or in lyophilized form. The container means of the kits may include at least one vial, test tube, flask, bottle, syringe, or other container means, into which a component may he placed, and preferably, suitably aliquoted. Where there is more than one component in the kit, the kit also will generally contain a second, third, or other additional containers into which the additional components may be separately placed. However, various combinations of components may be comprised in a vial. The kits of the present invention also will typically include a means for containing the approved product and any other reagent containers in close confinement for commercial sale. Such containers may include injection or blow molded plastic containers into which the desired vials are retained, for example.

In one embodiment, the vial or dispenser device is accompanied by instructions for administration. In one embodiment, the vial or dispenser is also accompanied with a notice associated with the container in form prescribed by a governmental agency regulating the manufacture, use, or sale of pharmaceuticals, which notice is reflective of approval by the agency of the form of the drug for human or veterinary administration. Such notice, for example, is the labeling approved by the U.S. Food and Drug Administration for prescription drugs, or the approved product insert. In one embodiment, compositions containing a compound provided herein formulated in a compatible pharmaceutical carrier are also prepared, placed in an appropriate container, and labeled for treatment of an indicated condition.

### Methods of Negotiating a Maximum Fair Price, Methods of Selling, and Methods of Offering for Sale an Approved Product

Provided herein are methods for negotiating a maximum fair price for an approved product that is negotiation eligible; for example, in accordance with H.R. 5376 (Inflation Reduction Act of 2022), which is incorporated by reference herein. An embodiment of the method comprises identifying the approved product as negotiation eligible, selecting said negotiation eligible approved product to be included in a price negotiation with a license holder of said approved product, wherein said license holder and a third party enter into negotiations to determine a maximum fair price for said negotiation eligible approved product, and concluding said negotiations with an agreed-upon maximum fair price for said negotiation eligible approved product. As used herein, the term “maximum fair price” means, with respect to a plan year during a price applicability period and with respect to a selected drug (as defined in section 1192 (c)) with respect to such period, the price published pursuant to section 1195 in the Federal Register for such drug and year.

Also provided herein are methods of selling an approved product, said method comprising selling such approved product, wherein an approved product label for a reference product for such approved product includes instructions for treating adult patients with relapsed or refractory multiple myeloma after at least four prior lines of therapy, including a proteasome inhibitor, an immunomodulatory agent, and an anti-CD38 monoclonal antibody. In some embodiments, the methods comprise selling teclistamab-cqyv.

In some embodiments, the methods of selling comprise selling the biosimilar, wherein an approved product label for a reference product for the biosimilar includes instructions for treating adult patients with relapsed or refractory multiple myeloma after at least four prior lines of therapy, including a proteasome inhibitor, an immunomodulatory agent, and an anti-CD38 monoclonal antibody.

Also provided herein are methods of offering for sale an approved product, said method comprising offering for sale such approved product, wherein an approved product label for a reference product for such approved product includes instructions for treating adult patients with relapsed or refractory multiple myeloma after at least four prior lines of therapy, including a proteasome inhibitor, an immunomodulatory agent, and an anti-CD38 monoclonal antibody. In some embodiments, the methods comprise offering teclistamab-cqyv for sale.

In some embodiments, the methods of offering for sale comprise offering for sale the biosimilar, wherein an approved product label for a reference product for the biosimilar includes instructions for treating adult patients with relapsed or refractory multiple myeloma after at least four prior lines of therapy, including a proteasome inhibitor, an immunomodulatory agent, and an anti-CD38 monoclonal antibody. In some embodiments, the biosimilar is a biosimilar of Teclistamab-cqyv.

Provided herein are methods of selling of kits. The method comprising selling such kits, wherein an approved product label for a reference product for such approved product includes instructions for treating multiple myeloma such as those described in Example 1. In some embodiments, the method comprises selling the kit, wherein an approved product label for a reference product for the approved product includes instructions for treating a patient with relapsed or refractory multiple myeloma after at least four prior lines of therapy, including a proteasome inhibitor, an immunomodulatory agent, and an anti-CD38 monoclonal antibody.

In some embodiments, the methods of selling comprise methods of selling a kit comprising a biosimilar, said method comprising selling the biosimilar, wherein an approved product label for a reference product for the biosimilar includes instructions for treating a patient with relapsed or refractory multiple myeloma, and in some embodiments, for treating a patient with relapsed or refractory multiple myeloma after at least four prior lines of therapy, including a proteasome inhibitor, an immunomodulatory agent, and an anti-CD38 monoclonal antibody.

The disclosure further provides methods of offering for sale a kit comprising teclistamab-cqyv, said method comprising offering for sale such kit, wherein the approved product label for a reference product for teclistamab-cqyv includes instructions for treating a patient with relapsed or refractory multiple myeloma, and in some embodiments, for treating a patient with relapsed or refractory multiple myeloma after at least four prior lines of therapy, including a proteasome inhibitor, an immunomodulatory agent and an anti-CD38 monoclonal antibody.

Also described herein are methods comprising selling a kit comprising a biosimilar or the approved product, wherein the approved product label for a biosimilar or the approved product comprises overall response rate data, duration of response data, duration of response rate, and/or time to first response. In some embodiments, the biosimilar label that is similar to or the same as a reference product label comprises overall response rate data. In some embodiments, the overall response rate data is stringent complete response data. In some embodiments, the overall response rate data is complete response data. In some embodiments, the overall response rate data is very good partial response data. In some embodiments, the overall response rate data is partial response data. An example of the overall response rate data is stringent complete response data, complete response data, very good partial response data, and partial response data.

In some embodiments, the overall response rate data for a biosimilar or the approved product is at least 61.8%, in particular, wherein the patients have relapsed or refractory multiple myeloma. In some embodiments, the overall response rate data for a biosimilar or the approved product is at least 61.8%, in particular, wherein the patients have relapsed or refractory multiple myeloma, who have received at least three prior therapies, including a proteasome inhibitor, immunomodulatory agent, and anti-CD38 monoclonal antibody.

In some embodiments, the complete response or better (stringent complete response (sCR)+complete response (CR)) data for a biosimilar or the approved product is at least 28.2%, in particular, wherein the patients have relapsed or refractory multiple myeloma, who have received at least three prior therapies, including a proteasome inhibitor, immunomodulatory agent, and anti-CD38 monoclonal antibody.

In some embodiments, the very good partial response data for a biosimilar or the approved product is at least 29.1%, in particular, wherein the patients have relapsed or refractory multiple myeloma, who have received at least three prior therapies, including a proteasome inhibitor, immunomodulatory agent, and anti-CD38 monoclonal antibody.

In some embodiments, the partial response data for a biosimilar or the approved product is at least 4.5%, in particular, wherein the patients have relapsed or refractory multiple myeloma, who have received at least three prior therapies, including a proteasome inhibitor, immunomodulatory agent, and anti-CD38 monoclonal antibody.

In some embodiments, the duration of response (DOR) data for a biosimilar or the approved product is at least 9 months, in particular, wherein the patients have relapsed or refractory multiple myeloma, who have received at least three prior therapies, including a proteasome inhibitor, immunomodulatory agent, and anti-CD38 monoclonal antibody.

In some embodiments, the time to first response data for a biosimilar or the approved product is at least 1.2 months, in particular, wherein the patients have relapsed or refractory multiple myeloma, who have received at least three prior therapies, including a proteasome inhibitor, immunomodulatory agent, and anti-CD38 monoclonal antibody.

In some embodiments, the estimated DOR rate data for a biosimilar or the approved product is at least 90.6% (95% CI: 80.3%, 95.7%) at 6 months and 66.5% (95% CI: 38.8%, 83.9%) at 9 months, in particular, wherein the patients have relapsed or refractory multiple myeloma, who have received at least three prior therapies, including a proteasome inhibitor, immunomodulatory agent, and anti-CD38 monoclonal antibody. For example, with a median follow-up of 7.4 months among responders, the estimated duration of response (DOR) rate can be 90.6% (95% CI: 80.3%, 95.7%) at 6 months and 66.5% (95% CI: 38.8%, 83.9%) at 9 months, wherein the patients have relapsed or refractory multiple myeloma, who have received at least three prior therapies, including a proteasome inhibitor, immunomodulatory agent, and anti-CD38 monoclonal antibody.

Still further provided herein are methods of improving overall response rate, duration of response, and time to first response in patients with relapsed or refractory multiple myeloma, said methods comprising administering to said patients a biosimilar or the approved product. Also provided herein are methods of improving overall response rate data in patients with relapsed or refractory multiple myeloma, said methods comprising administering to said patients a biosimilar or the approved product. Also provided herein are methods of improving complete response or better data in patients with relapsed or refractory multiple myeloma, said methods comprising administering to said patients a biosimilar or the approved product. Also provided herein are methods of improving very good partial response data in patients with relapsed or refractory multiple myeloma, said methods comprising administering to said patients a biosimilar or the approved product. Also provided herein are methods of improving partial response data in patients with relapsed or refractory multiple myeloma, said methods comprising administering to said patients a biosimilar or the approved product. Also provided herein are methods of improving duration of response data in patients with relapsed or refractory multiple myeloma, said methods comprising administering to said patients a biosimilar or the approved product. Also provided herein are methods of improving time to first response data in patients with relapsed or refractory multiple myeloma, said methods comprising administering to said patients a biosimilar or the approved product. Also provided herein are methods of improving duration of response rate data in patients with relapsed or refractory multiple myeloma, said methods comprising administering to said patients a biosimilar or the approved product.

In some embodiments, the overall response rate data for a biosimilar or the approved product is at least 61.8%, in particular, wherein the patients have relapsed or refractory multiple myeloma. In some embodiments, the overall response rate data for a biosimilar or the approved product is at least 61.8%, in particular, wherein the patients have relapsed or refractory multiple myeloma, who have received at least three prior therapies, including a proteasome inhibitor, immunomodulatory agent, and anti-CD38 monoclonal antibody. In some embodiments, the complete response or better data for a biosimilar or the approved product is at least 28.2%, in particular, wherein the patients have relapsed or refractory multiple myeloma, who have received at least three prior therapies, including a proteasome inhibitor, immunomodulatory agent, and anti-CD38 monoclonal antibody. In some embodiments, the very good partial response data for a biosimilar or the approved product is at least 29.1%, in particular, wherein the patients have relapsed or refractory multiple myeloma, who have received at least three prior therapies, including a proteasome inhibitor, immunomodulatory agent, and anti-CD38 monoclonal antibody. In some embodiments, the partial response data for a biosimilar or the approved product is at least 4.5%, in particular, wherein the patients have relapsed or refractory multiple myeloma, who have received at least three prior therapies, including a proteasome inhibitor, immunomodulatory agent, and anti-CD38 monoclonal antibody. In some embodiments, the duration of response data for a biosimilar or approved product is at least 9 months, in particular, wherein the patients have relapsed or refractory multiple myeloma, who have received at least three prior therapies, including a proteasome inhibitor, immunomodulatory agent, and anti-CD38 monoclonal antibody. In some embodiments, the time to first response data for a biosimilar or the approved product is at least 1.2 months, in particular, wherein the patients have relapsed or refractory multiple myeloma, who have received at least three prior therapies, including a proteasome inhibitor, immunomodulatory agent, and anti-CD38 monoclonal antibody. In some embodiments, the duration of response rate data for a biosimilar or approved product is at least 90.6% (95% CI: 80.3%, 95.7%) at 6 months and 66.5% (95% CI: 38.8%, 83.9%) at 9 months, in particular, wherein the patients have relapsed or refractory multiple myeloma, who have received at least three prior therapies, including a proteasome inhibitor, immunomodulatory agent, and anti-CD38 monoclonal antibody.

### EXAMPLES

These examples are provided for illustrative purposes only and not to limit the scope of the claims provided herein.

### Example 1: Approved Drug Product Label

### Highlights of Prescribing Information

These highlights do not include all the information needed to use TECVAYLI safely and effectively. See full prescribing information for TECVAYLI.
TECVAYLI™ (teclistamab-cqyv) injection, for subcutaneous use

### Initial U.S. Approval: 2022

### Warning: CYTOKINE RELEASE SYNDROME and NEUROLOGIC TOXICITY Including Immune Effector Cell-Associated Neurotoxicity Syndrome

### See Full Prescribing Information for Complete Boxed Warning

Cytokine release syndrome (CRS), including life-threatening or fatal reactions, can occur in patients receiving TECVAYLI. Initiate treatment with TECVAYLI step-up dosing schedule to reduce risk of CRS. Withhold TECVAYLI until CRS resolves or permanently discontinue based on severity. (2.1, 2.4, 5.1)
Neurologic toxicity, including Immune Effector Cell-Associated Neurotoxicity Syndrome (ICANS) and serious and life-threatening reactions, can occur in patients receiving TECVAYLI. Monitor patients for signs or symptoms of neurologic toxicity, including ICANS, during treatment. Withhold TECVAYLI until neurologic toxicity resolves or permanently discontinue based on severity. (2.4, 5.2)
TECVAYLI is available only through a restricted program called the TECVAYLI Risk Evaluation and Mitigation Strategy (REMS). (5.3)

### Indications and Usage

TECVAYLI is a bispecific B-cell maturation antigen (BCMA)-directed CD3 T-cell engager indicated for the treatment of adult patients with relapsed or refractory multiple myeloma who have received at least four prior lines of therapy, including a proteasome inhibitor, an immunomodulatory agent and an anti-CD38 monoclonal antibody.
This indication is approved under accelerated approval based on response rate. Continued approval for this indication may be contingent upon verification and description of clinical benefit in confirmatory trial(s). (1)

### Dosage and Administration

TECVAYLI Recommended Dosing Schedule (2.1)

| | | |
|---|---|---|
| Dosing | | |
| Schedule | Day | Dose |

| | | | |
|---|---|---|---|
| Step-up | Day 1 | Step-up | 0.06 mg/kg |
| Dosing | | dose 1 | |
| Schedule | Day 4 | Step-up | 0.3 mg/kg |
| | | dose 2 | |
| | Day 7 | First | 1.5 mg/kg |
| | | treatment | |
| | | dose | |
| Weekly | One week after | Subsequent | 1.5 mg/kg |
| Dosing | first treatment | treatment | once |
| Schedule | dose and weekly | doses | weekly |
| | thereafter | | |

For subcutaneous injection only. (2.1)
Patients should be hospitalized for 48 hours after administration of all doses within the TECVAYLI step-up dosing schedule. (2.1)
Administer pretreatment medications as recommended. (2.2)
See Full Prescribing Information for instructions on preparation and administration. (2.5)

### Dosage Forms and Strengths

### Injection

30 mg/3 mL (10 mg/mL) in a single-dose vial (3)
153 mg/1.7 mL (90 mg/mL) in a single-dose vial (3)

### Contraindications

### None. (4)

### Warnings and Precautions

Hepatotoxicity: Can cause hepatotoxicity, including fatalities. Monitor liver enzymes and bilirubin at baseline and during treatment as clinically indicated. (5.4)
Infections: Can cause severe, life-threatening, or fatal infections. Monitor patients for signs and symptoms of infection and treat appropriately. Withhold in patients with active infection during the step-up dosing schedule. (2.4, 5.5)
Neutropenia: Monitor complete blood cell counts at baseline and periodically during treatment. (5.6)
Hypersensitivity and Other Administration Reactions: Systemic administration-related reactions and local injection site reactions can occur. Withhold or consider permanent discontinuation based on severity. (2.4, 5.7)
Embryo-Fetal Toxicity: May cause fetal harm. Advise females of reproductive potential of the potential risk to the fetus and to use effective contraception. (5.8, 8.1, 8.3)

### Adverse Reactions

The most common adverse reactions (>20%) are pyrexia, cytokine release syndrome, musculoskeletal pain, injection site reaction, fatigue, upper respiratory tract infection, nausea, headache, pneumonia, and diarrhea.
The most common Grade 3 to 4 laboratory abnormalities (>20%) are decreased lymphocytes, decreased neutrophils, decreased white blood cells, decreased hemoglobin, and decreased platelets. (6.1)
To report SUSPECTED ADVERSE REACTIONS, contact Janssen Biotech, Inc. at 1-800-JANSSEN (1-800-526-7736) or FDA at 1-800-FDA-1088 or www.fda.gov/mechvatch.

### Use in Specific Populations

Lactation: Advise not to breastfeed. (8.2)

See 17 for PATIENT COUNSELING INFORMATION and Medication Guide.

### Full Prescribing Information: Contents*

### Warning: Cytokine Release Syndrome and Neurologic Toxicity Including Immune Effector Cell-Associated Neurotoxicity Syndrome

1 INDICATIONS AND USAGE
2 DOSAGE AND ADMINISTRATION
   2.1 Recommended Dosage
   2.2 Recommended Pretreatment Medications
   2.3 Restarting TECVAYLI after Dosage Delay
   2.4 Dosage Modifications for Adverse Reactions
   2.5 Preparation and Administration
3 DOSAGE FORMS AND STRENGTHS
4 CONTRAINDICATIONS
5 WARNINGS AND PRECAUTIONS
   5.1 Cytokine Release Syndrome
   5.2 Neurologic Toxicity including ICANS
   5.3 TECVAYLI REMS
   5.4 Hepatotoxicity
   5.5 Infections
   5.6 Neutropenia
   5.7 Hypersensitivity and Other Administration Reactions
   5.8 Embryo-Fetal Toxicity
6 ADVERSE REACTIONS
   6.1 Clinical Trials Experience
7 DRUG INTERACTIONS
8 USE IN SPECIFIC POPULATIONS
   8.1 Pregnancy
   8.2 Lactation
   8.3 Females and Males of Reproductive Potential
   8.4 Pediatric Use
   8.5 Geriatric Use
11 DESCRIPTION
12 CLINICAL PHARMACOLOGY
   12.1 Mechanism of Action
   12.2 Pharmacodynamics
   12.3 Pharmacokinetics
   12.6 Immunogenicity
13 NONCLINICAL TOXICOLOGY
   13.1 Carcinogenesis, Mutagenesis, Impairment of Fertility
14 CLINICAL STUDIES
16 HOW SUPPLIED/STORAGE AND HANDLING
17 PATIENT COUNSELING INFORMATION
   *Sections or subsections omitted from the full prescribing information are not listed.

### Full Prescribing Information

WARNING: CYTOKINE RELEASE SYNDROME and NEUROLOGIC TOXICITY including IMMUNE EFFECTOR CELL-ASSOCIATED NEUROTOXICITY SYNDROME
Cytokine release syndrome (CRS), including life-threatening or fatal reactions, can occur in patients receiving TECVAYLI. Initiate treatment with TECVAYLI step-up dosing schedule to reduce risk of CRS. Withhold TECVAYLI until CRS resolves or permanently discontinue based on severity [see Dosage and Administration (2.1, 2.4) and Warnings and Precautions (5.1)].
Neurologic toxicity, including Immune Effector Cell-Associated Neurotoxicity Syndrome (ICANS) and serious and life-threatening reactions, can occur with TECVAYLI. Monitor patients for signs or symptoms of neurologic toxicity, including ICANS, during treatment. Withhold TECVAYLI until neurologic toxicity resolves or permanently discontinue based on severity [see Dosage and Administration (2.4) and Warnings and Precautions (5.2)].
Because of the risk of CRS and neurologic toxicity, including ICANS, TECVAYLI is available only through a restricted program under a Risk Evaluation and Mitigation Strategy (REMS) called the TECVAYLI REMS [see Warnings and Precautions (5.3)].

### 1 Indications and Usage

TECVAYLI is indicated for the treatment of adult patients with relapsed or refractory multiple myeloma who have received at least four prior lines of therapy, including a proteasome inhibitor, an immunomodulatory agent and an anti-CD38 monoclonal antibody.
This indication is approved under accelerated approval based on response rate [see Clinical Studies (14)]. Continued approval for this indication may be contingent upon verification and description of clinical benefit in confirmatory trial(s).

### 2 Dosage and Administration

### 2.1 Recommended Dosage

For subcutaneous injection only.
The recommended dosing schedule for TECVAYLI is provided in Table 1. The recommended dosage of TECVAYLI is step-up doses of 0.06 mg/kg and 0.3 mg/kg followed by 1.5 mg/kg once weekly until disease progression or unacceptable toxicity.
Administer pretreatment medications prior to each dose of the TECVAYLI step-up dosing schedule, which includes step-up dose 1, step-up dose 2, and the first treatment dose as described in Table 1 [see Dosage and Administration (2.2)].
Administer TECVAYLI subcutaneously according to the step-up dosing schedule in Table 1 to reduce the incidence and severity of cytokine release syndrome (CRS). Due to the risk of CRS and neurologic toxicity, including ICANS, patients should be hospitalized for 48 hours after administration of all doses within the TECVAYLI step-up dosing schedule [see Dosage and Administration (2.4) and Warnings and Precautions (5.1, 5.2)].

TABLE 1
TECVAYLI Dosing Schedule

Dosing

| schedule | Day | Dose | |
|---|---|---|---|
| Step-up dosing | Day 1 | Step-up dose 1 | 0.06 mg/kg |
| schedule^{a} | Day 4^{b} | Step-up dose 2 | 0.3 mg/kg |
| | Day 7^{c} | First treatment dose | 1.5 mg/kg |
| Weekly dosing | One week after first | Subsequent | 1.5 mg/kg |
| schedule^{a} | treatment dose and | treatment doses | once |
| | weekly thereafter | | weekly |

^{a}See Table 2 for recommendations on restarting TECVAYLI after dose delays [see Dosage and Administration (2.3)].
^{b}Step-up dose 2 may be given between 2 to 4 days after step-up dose 1 and may be given up to 7 days after step-up dose 1 to allow for resolution of adverse reactions.
^{c}First treatment dose may be given between 2 to 4 days after step-up dose 2 and may be given up to 7 days after step-up dose 2 to allow for resolution of adverse reactions.

### 2.2 Recommended Pretreatment Medications

Administer the following pretreatment medications 1 to 3 hours before each dose of the TECVAYLI step-up dosing schedule, which includes step-up dose 1, step-up dose 2, and the first treatment dose (see Table 1), to reduce the risk of CRS [see Warnings and Precautions (5.1) and Adverse Reactions (6.1)].
Corticosteroid (oral or intravenous dexamethasone 16 mg)
Histamine-1 (H1) receptor antagonist (oral or intravenous diphenhydramine 50 mg or equivalent)
Antipyretics (oral or intravenous acetaminophen 650 mg to 1,000 mg or equivalent)
   Administration of pretreatment medications may be required prior to administration of subsequent doses of TECVAYLI in the following patients:
Patients who repeat doses within the TECVAYLI step-up dosing schedule following a dose delay [see Dosage and Administration (2.3)].
Patients who experienced CRS following the prior dose of TECVAYLI [see Dosage and Administration (2.4)].

### Prophylaxis for Herpes Zoster Reactivation

Prior to starting treatment with TECVAYLI, consider initiation of antiviral prophylaxis to prevent herpes zoster reactivation per guidelines.

2.3 Restarting TECVAYLI after Dosage Delay
If a dose of TECVAYLI is delayed, restart therapy based on the recommendations in Table 2 and resume the treatment schedule accordingly [see Dosage and Administration (2.1)].
Administer pretreatment medications as indicated in Table 2. Due to the risk of CRS and neurologic toxicity, including ICANS, patients should be hospitalized for 48 hours after administration of all doses within the TECVAYLI step-up dosing schedule [see Dosage and Administration (2.4) and Warnings and Precautions (5.1, 5.2)].

TABLE 2
Recommendations for Restarting Therapy
with TECVAYLI After Dose Delay

| | Duration of | |
|---|---|---|
| | delay from | |
| Last dose | the last dose | |
| administered | administered | Action |
| Step-up dose 1 | More than | Restart TECVAYLI step-up dosing |
| | 7 days | schedule at step-up dose 1 (0.06 mg/kg).^{a} |
| Step-up dose 2 | 8 days to | Repeat step-up dose 2 (0.3 mg/kg)^{a} |
| | 28 days | and continue TECVAYLI step-up |
| | | dosing schedule. |
| | More than | Restart TECVAYLI step-up dosing |
| | 28 days^{b} | schedule at step-up dose 1 (0.06 mg/kg).^{a} |
| Any treatment | 8 days to | Continue TECVAYLI weekly dosing |
| dose | 28 days | schedule at treatment dose (1.5 mg/kg). |
| | More than | Restart TECVAYLI step-up dosing |
| | 28 days^{b} | schedule at step-up dose 1 (0.06 mg/kg).^{a} |

^{a}Administer pretreatment medications prior to TECVAYLI dose and monitor patients accordingly [see Dosage and Administration (2.2, 2.5)].
^{b}Consider benefit-risk of restarting TECVAYLI in patients who require a dose delay of more than 28 days due to an adverse reaction.

### 2.4 Dosage Modifications for Adverse Reactions

Dosage reductions of TECVAYLI are not recommended.
Dosage delays may be required to manage toxicities related to TECVAYLI [see Warnings and Precautions (5)].
See Tables 3, 4, and 5 for recommended actions for adverse reactions of CRS, neurologic toxicity, and ICANS. See Table 6 for recommended actions for other adverse reactions following administration of TECVAYLI.

### Management of CRS, Neurologic Toxicity and ICANS

### Cytokine Release Syndrome (CRS)

Management recommendations for CRS are summarized in Table 3.
Identify CRS based on clinical presentation [see Warnings and Precautions (5.1)]. Evaluate and treat other causes of fever, hypoxia, and hypotension.
If CRS is suspected, withhold TECVAYLI until CRS resolves. Manage according to the recommendations in Table 3 and consider further management per current practice guidelines. Administer supportive therapy for CRS, which may include intensive care for severe or life-threatening CRS. Consider laboratory testing to monitor for disseminated intravascular coagulation (DIC), hematology parameters, as well as pulmonary, cardiac, renal, and hepatic function.

TABLE 3
Recommendations for Management of Cytokine Release Syndrome

| Grade^{a} | Presenting Symptoms | Actions |
|---|---|---|
| Grade 1 | Temperature ≥100.4° F. (38° C.)^{b} | Withhold TECVAYLI until CRS resolves. |
| | | Administer pretreatment medications prior |
| | | to next dose of TECVAYLI.^{c} |
| Grade 2 | Temperature ≥100.4° F. (38° C.)^{b} | Withhold TECVAYLI until CRS resolves. |
| | with: | Administer pretreatment medications prior |
| | Hypotension responsive to fluids | to next dose of TECVAYLI.^{c} |
| | and not requiring vasopressors, | Patients should be hospitalized for |
| | and/or, | 48 hours following the next dose of |
| | Oxygen requirement of low-flow | TECVAYLI [see Dosage and |
| | nasal cannula^{d} or blow-by. | Administration (2.1)].^{c} |
| Grade 3 | Temperature ≥100.4° F. (38° C.)^{b} | First Occurrence of Grade 3 CRS with |
| | with: | Duration Less than 48 Hours: |
| | Hypotension requiring one | Withhold TECVAYLI until CRS resolves. |
| | vasopressor with or without | Provide supportive therapy, which may |
| | vasopressin, | include intensive care. |
| | and/or, | Administer pretreatment medications prior |
| | Oxygen requirement of high-flow | to next dose of TECVAYLI.^{c} |
| | nasal cannula^{a}, facemask, non- | Patients should be hospitalized for |
| | rebreather mask, or Venturi | 48 hours following the next dose of |
| | mask. | TECVAYLI [see Dosage and |
| | | Administration (2.1)].^{c} |
| | | Recurrent Grade 3 CRS or Grade 3 CRS with |
| | | Duration 48 Hours or Longer: |
| | | Permanently discontinue TECVAYLI. |
| | | Provide supportive therapy, which may |
| | | include intensive care. |
| Grade 4 | Temperature ≥100.4° F. (38° C.)^{b} | Permanently discontinue TECVAYLI. |
| | with: | Provide supportive therapy, which may |
| | Hypotension requiring multiple | include intensive care. |
| | vasopressors (excluding | |
| | vasopressin), | |
| | and/or, | |
| | Oxygen requirement of positive | |
| | pressure (e.g., continuous | |
| | positive airway pressure (CPAP), | |
| | bilevel positive airway pressure | |
| | (BiPAP), intubation, and | |
| | mechanical ventilation). | |

^{a}Based on American Society for Transplantation and Cellular Therapy (ASTCT) 2019 grading for CRS.
^{b}Attributed to CRS. Fever may not always be present concurrently with hypotension or hypoxia as it may be masked by interventions such as antipyretics or anticytokine therapy.
^{c}See Table 2 for recommendations on restarting TECVAYLI after dose delays [see Dosage and Administration (2.3)].
^{d}Low-flow nasal cannula is ≤6 L/min, and high-flow nasal cannula is >6 L/min.

### Neurologic Toxicity and ICANS

Management recommendations for neurologic toxicity and ICANS are summarized in Tables 4 and 5.
At the first sign of neurologic toxicity, including ICANS, withhold TECVAYLI and consider neurology evaluation. Rule out other causes of neurologic symptoms. Provide supportive therapy, which may include intensive care, for severe or life-threatening neurologic toxicities, including ICANS [see Warnings and Precautions (5.2)]. Manage ICANS according to the recommendations in Table 5 and consider further management per current practice guidelines.

TABLE 4
Recommendations for Management of Neurologic
Toxicity (excluding ICANS)

| Adverse | | |
|---|---|---|
| Reaction | Severity^{a} | Actions |
| Neurologic | Grade 1 | Withhold TECVAYLI until |
| Toxicity^{a} | | neurologic toxicity symptoms |
| (excluding | | resolve or stabilize.^{b} |
| ICANS) | Grade 2 | Withhold TECVAYLI until |
| | Grade 3 (First | neurologic toxicity symptoms |
| | occurrence) | improve to Grade 1 or less.^{b} |
| | | Provide supportive therapy. |
| | Grade 3 (Recurrent) | Permanently discontinue |
| | Grade 4 | TECVAYLI. |
| | | Provide supportive therapy, which |
| | | may include intensive care. |

^{a}Based on National Cancer Institute Common Terminology Criteria for Adverse Events (NCI CTCAE), version 4.03.
^{b}See Table 2 for recommendations on restarting TECVAYLI after dose delays [see Dosage and Administration (2.3)].

TABLE 5
Recommendations for Management of Immune Effector
Cell-Associated Neurotoxicity Syndrome

| Grade^{a} | Presenting Symptoms^{b} | Actions |
|---|---|---|
| Grade 1 | ICE score 7-9^{c}, | Withhold TECVAYLI until |
| | or depressed level of | ICANS resolves.^{e} |
| | consciousness^{d}: awakens | Monitor neurologic symptoms |
| | spontaneously. | and consider consultation with |
| | | neurologist and other specialists |
| | | for further evaluation and |
| | | management, including |
| | | consideration for starting non- |
| | | sedating, anti-seizure medicines |
| | | for seizure prophylaxis. |
| Grade 2 | ICE score 3-6^{c}, | Withhold TECVAYLI until |
| | or depressed level of | ICANS resolves. |
| | consciousness^{d}: awakens | Administer dexamethasone^{f} |
| | to voice. | 10 mg intravenously every |
| | | 6 hours. Continue |
| | | dexamethasone use until |
| | | resolution to Grade 1 or less |
| | | then taper. |
| | | Monitor neurologic symptoms |
| | | and consider consultation with |
| | | neurologist and other specialists |
| | | for further evaluation and |
| | | management, including |
| | | consideration for starting non- |
| | | sedating, anti-seizure medicines |
| | | for seizure prophylaxis. |
| | | Patients should be hospitalized |
| | | for 48 hours following the next |
| | | dose of TECVAYLI [see Dosage |
| | | and Administration (2.1)].^{e} |
| Grade 3 | ICE score 0-2^{c}, | First Occurrence of Grade 3 |
| | or depressed level of | ICANS: |
| | consciousness^{d}: | Withhold TECVAYLI until |
| | awakens only to | ICANS resolves. |
| | tactile stimulus, | Administer dexamethasone^{f} |
| | or seizures^{d}, either: | 10 mg intravenously every |
| | any clinical seizure, | 6 hours. Continue |
| | focal or generalized, | dexamethasone use until |
| | that resolves rapidly, or | resolution to Grade 1 or less, |
| | non-convulsive seizures on | then taper. |
| | electroencephalogram | Monitor neurologic symptoms |
| | (EEG) that resolve with | and consider consultation with |
| | intervention, or raised | neurologist and other specialists |
| | intracranial pressure: | for further evaluation and |
| | focal/local edema on | management, including |
| | neuroimaging^{d}. | consideration for starting non- |
| | | sedating, anti-seizure medicines |
| | | for seizure prophylaxis. |
| | | Provide supportive therapy, |
| | | which may include intensive |
| | | care. |
| | | Patients should be hospitalized |
| | | for 48 hours following the next |
| | | dose of TECVAYLI [see Dosage |
| | | and Administration (2.1)].^{e} |
| | | Recurrent Grade 3 ICANS: |
| | | Permanently discontinue |
| | | TECVAYLI |
| | | Administer dexamethasone^{f} |
| | | 10 mg intravenously and repeat |
| | | dose every 6 hours. Continue |
| | | dexamethasone use until |
| | | resolution to Grade 1 or less, |
| | | then taper. |
| | | Monitor neurologic symptoms |
| | | and consider consultation with |
| | | neurologist and other specialists |
| | | for further evaluation and |
| | | management, including |
| | | consideration for starting non- |
| | | sedating, anti-seizure medicines |
| | | for seizure prophylaxis. |
| | | Provide supportive therapy, |
| | | which may include intensive |
| | | care. |
| Grade 4 | ICE score 0^{c}, | Permanently discontinue |
| | or depressed level of | TECVAYLI. |
| | consciousness^{d}: either: | Administer dexamethasone^{f} |
| | patient is unarousable or | 10 mg intravenously and repeat |
| | requires vigorous or | dose every 6 hours. Continue |
| | repetitive tactile | dexamethasone use until |
| | stimuli to arouse, or | resolution to Grade 1 or less, |
| | stupor or coma, | then taper. |
| | or seizures^{d}, either: | Alternatively, consider |
| | life-threatening prolonged | administration of |
| | seizure (>5 minutes), or | methylprednisolone 1,000 mg per |
| | repetitive clinical or | day intravenously and continue |
| | electrical seizures | methylprednisolone 1,000 mg per |
| | without return to | day intravenously for |
| | baseline in between, | 2 or more days. |
| | or motor findings^{d}: | Monitor neurologic symptoms |
| | deep focal motor weakness | and consider consultation with |
| | such as hemiparesis or | neurologist and other specialists |
| | paraparesis, or raised | for further evaluation and |
| | intracranial pressure/ | management, including |
| | cerebral edema^{d}, with | consideration for starting non- |
| | signs/symptoms such as: | sedating, anti-seizure medicines |
| | diffuse cerebral edema on | for seizure prophylaxis. |
| | neuroimaging, or | Provide supportive therapy, |
| | decerebrate or decorticate | which may include intensive |
| | posturing, or | care. |
| | cranial nerve VI palsy, or | |
| | papilledema, or | |
| | Cushing's triad. | |

^{a}Based on American Society for Transplantation and Cellular Therapy (ASTCT) 2019 grading for ICANS.
^{b}Management is determined by the most severe event, not attributable to any other cause.
^{c}If patient is arousable and able to perform Immune Effector Cell-Associated Encephalopathy (ICE) Assessment, assess: Orientation (oriented to year, month, city, hospital = 4 points); Naming (name 3 objects, e.g., point to clock, pen, button = 3 points); Following Commands (e.g., “show me 2 fingers” or “close your eyes and stick out your tongue” = 1 point); Writing (ability to write a standard sentence = 1 point; and Attention (count backwards from 100 by ten = 1 point). If patient is unarousable and unable to perform ICE Assessment (Grade 4 ICANS) = 0 points.
^{d}Not attributable to any other cause.
^{e}See Table 2 for recommendations on restarting TECVAYLI after dose delays [see Dosage and Administration (2.3)].
^{f}All references to dexamethasone administration are dexamethasone or equivalent.

TABLE 6
Recommended Dosage Modifications for Other Adverse Reactions

| Adverse | | |
|---|---|---|
| Reactions | Severity | Actions |
| Infections^{a} | All Grades | Withhold TECVAYLI in |
| [see Warnings | | patients with active infection |
| and Precautions | | during the step-up dosing |
| (5.5)] | | schedule.^{b} |
| | Grade 3 | Withhold subsequent treatment |
| | | doses of TECVAYLI (i.e., doses |
| | | administered after TECVAYLI |
| | | step-up dosing schedule) until |
| | | infection improves to Grade 1 or |
| | | less.^{b} |
| | Grade 4 | Consider permanent |
| | | discontinuation of TECVAYLI. |
| | | If TECVAYLI is not |
| | | permanently discontinued, |
| | | withhold subsequent treatment |
| | | doses of TECVAYLI (i.e., doses |
| | | administered after TECVAYLI |
| | | step-up dosing schedule) until |
| | | infection improves to Grade 1 or |
| | | less.^{b} |
| Hematologic | Absolute neutrophil | Withhold TECVAYLI until |
| Toxicities | count less than | absolute neutrophil count is |
| [see Warnings | 0.5 Í 10⁹/L | 0.5 Í 10⁹/L or higher.^{b} |
| and Precautions | Febrile neutropenia | Withhold TECVAYLI until |
| (5.6) and Adverse | | absolute neutrophil count is |
| Reactions (6.1)] | | 1 Í 10⁹/L or higher and fever |
| | | resolves.^{b} |
| | Hemoglobin less | Withhold TECVAYLI until |
| | than 8 g/dL | hemoglobin is 8 g/dL or higher.^{b} |
| | Platelet count less | Withhold TECVAYLI until |
| | than 25,000/mcL | platelet count is 25,000/mcL or |
| | Platelet count | higher and no evidence of |
| | between | bleeding.^{b} |
| | 25,000/mcL and | |
| | 50,000/mcL with | |
| | bleeding | |
| Other Non- | Grade 3 | Withhold TECVAYLI until |
| Hematologic | | adverse reaction improves to |
| Adverse | | Grade 1 or less.^{b} |
| Reactions^{a} | Grade 4 | Consider permanent |
| [see Warnings | | discontinuation of TECVAYLI. |
| and Precautions | | If TECVAYLI is not |
| (5.4) and Adverse | | permanently discontinued, |
| Reactions (6.1)] | | withhold subsequent treatment |
| | | doses of TECVAYLI (i.e., doses |
| | | administered after TECVAYLI |
| | | step-up dosing schedule) until |
| | | adverse reaction improves to |
| | | Grade 1 or less.^{b} |

^{a}Based on National Cancer Institute Common Terminology Criteria for Adverse Events (NCI CTCAE), version 4.03.
^{b}See Table 2 for recommendations on restarting TECVAYLI after dose delays [see Dosage and Administration (2.3)].

### 2.5 Preparation and Administration

TECVAYLI is intended for subcutaneous use by a healthcare provider only.
TECVAYLI should be administered by a healthcare provider with adequate medical personnel and appropriate medical equipment to manage severe reactions, including CRS and ICANS [see Warnings and Precautions (5.1, 5.2)].
TECVAYLI is a clear to slightly opalescent, colorless to light yellow solution. Parenteral drug products should be inspected visually for particulate matter and discoloration prior to administration, whenever solution and container permit. Do not use if the solution is discolored, or cloudy, or if foreign particles are present.
TECVAYLI 30 mg/3 mL (10 mg/mL) vial and TECVAYLI 153 mg/1.7 mL (90 mg/mL) vial are supplied as ready-to-use solution that do not need dilution prior to administration.
Do not combine TECVAYLI vials of different concentrations to achieve treatment dose.
Use aseptic technique to prepare and administer TECVAYLI.

### Preparation of TECVAYLI

Refer to the following reference tables for the preparation of TECVAYLI.
Use Table 7 to determine total dose, injection volume and number of vials required based on patient's actual body weight for step-up dose 1 using TECVAYLI 30 mg/3 mL (10 mg/mL) vial.

TABLE 7
Step-up Dose 1 (0.06 mg/kg) Injection Volumes
using TECVAYLI 30 mg/3 mL (10 mg/mL) Vial

| Patient Body | | | |
|---|---|---|---|
| Weight | Total Dose | Volume of Injection | Number of Vials |
| (kg) | (mg) | (mL) | (1 vial = 3 mL) |
| 35 to 39 | 2.2 | 0.22 | 1 |
| 40 to 44 | 2.5 | 0.25 | 1 |
| 45 to 49 | 2.8 | 0.28 | 1 |
| 50 to 59 | 3.3 | 0.33 | 1 |
| 60 to 69 | 3.9 | 0.39 | 1 |
| 70 to 79 | 4.5 | 0.45 | 1 |
| 80 to 89 | 5.1 | 0.51 | 1 |
| 90 to 99 | 5.7 | 0.57 | 1 |
| 100 to 109 | 6.3 | 0.63 | 1 |
| 110 to 119 | 6.9 | 0.69 | 1 |
| 120 to 129 | 7.5 | 0.75 | 1 |
| 130 to 139 | 8.1 | 0.81 | 1 |
| 140 to 149 | 8.7 | 0.87 | 1 |
| 150 to 160 | 9.3 | 0.93 | 1 |

Use Table 8 to determine total dose, injection volume and number of vials required based on patient's actual body weight for step-up dose 2 using TECVAYLI 30 mg/3 mL (10 mg/mL) vial.

TABLE 8
Step-up Dose 2 (0.3 mg/kg) Injection Volumes
using TECVAYLI 30 mg/3 mL (10 mg/mL) Vial

| Patient Body | | | |
|---|---|---|---|
| Weight | Total Dose | Volume of Injection | Number of Vials |
| (kg) | (mg) | (mL) | (1 vial = 3 mL) |
| 35 to 39 | 11 | 1.1 | 1 |
| 40 to 44 | 13 | 1.3 | 1 |
| 45 to 49 | 14 | 1.4 | 1 |
| 50 to 59 | 16 | 1.6 | 1 |
| 60 to 69 | 19 | 1.9 | 1 |
| 70 to 79 | 22 | 2.2 | 1 |
| 80 to 89 | 25 | 2.5 | 1 |
| 90 to 99 | 28 | 2.8 | 1 |
| 100 to 109 | 31 | 3.1 | 2 |
| 110 to 119 | 34 | 3.4 | 2 |
| 120 to 129 | 37 | 3.7 | 2 |
| 130 to 139 | 40 | 4 | 2 |
| 140 to 149 | 43 | 4.3 | 2 |
| 150 to 160 | 47 | 4.7 | 2 |

Use Table 9 to determine total dose, injection volume and number of vials required based on patient's actual body weight for the treatment dose using TECVAYLI 153 mg/1.7 mL (90 mg/mL) vial.

TABLE 9
Treatment Dose (1.5 mg/kg) Injection Volumes
using TECVAYLI 153 mg/1.7 mL (90 mg/mL) Vial

| Patient Body | | | |
|---|---|---|---|
| Weight | Total Dose | Volume of Injection | Number of Vials |
| (kg) | (mg) | (mL) | (1 vial = 1.7 mL) |
| 35 to 39 | 56 | 0.62 | 1 |
| 40 to 44 | 63 | 0.7 | 1 |
| 45 to 49 | 70 | 0.78 | 1 |
| 50 to 59 | 82 | 0.91 | 1 |
| 60 to 69 | 99 | 1.1 | 1 |
| 70 to 79 | 108 | 1.2 | 1 |
| 80 to 89 | 126 | 1.4 | 1 |
| 90 to 99 | 144 | 1.6 | 1 |
| 100 to 109 | 153 | 1.7 | 1 |
| 110 to 119 | 171 | 1.9 | 2 |
| 120 to 129 | 189 | 2.1 | 2 |
| 130 to 139 | 198 | 2.2 | 2 |
| 140 to 149 | 216 | 2.4 | 2 |
| 150 to 160 | 234 | 2.6 | 2 |

Remove the appropriate strength TECVAYLI vial from refrigerated storage [2° C. to 8° C. (36° F. to 46° F.)].
Once removed from refrigerated storage, equilibrate TECVAYLI to ambient temperature [15° C. to 30° C. (59° F. to 86° F.)] for at least 15 minutes. Do not warm TECVAYLI in any other way.
Once equilibrated, gently swirl the vial for approximately 10 seconds to mix. Do not shake.
Withdraw the required injection volume of TECVAYLI from the vial(s) into an appropriately sized syringe using a transfer needle.
Each injection volume should not exceed 2 mL. Divide doses requiring greater than 2 mL equally into multiple syringes.
TECVAYLI is compatible with stainless steel injection needles and polypropylene or polycarbonate syringe material.
Replace the transfer needle with an appropriately sized needle for injection.

### Administration of TECVAYLI

Inject the required volume of TECVAYLI into the subcutaneous tissue of the abdomen (preferred injection site). Alternatively, TECVAYLI may be injected into the subcutaneous tissue at other sites (e.g., thigh). If multiple injections are required, TECVAYLI injections should be at least 2 cm apart.
Do not inject into tattoos or scars or areas where the skin is red, bruised, tender, hard or not intact.
Any unused product or waste material should be disposed in accordance with local requirements.

### Storage

If the prepared dosing syringe(s) of TECVAYLI is not used immediately, store syringe(s) at 2° C. to 8° C. (36° F. to 46° F.) or at ambient temperature 15° C. to 30° C. (59° F. to 86° F.) for a maximum of 20 hours. Discard syringe(s) after 20 hours, if not used.

### Monitoring

Due to the risk of CRS and neurologic toxicity, including ICANS, patients should be hospitalized for 48 hours after administration of all doses within the TECVAYLI step-up dosing schedule [see Dosage and Administration (2.1) and Warnings and Precautions (5.1, 5.2)].

### 3 Dosage Forms and Strengths

### Injection

30 mg/3 mL (10 mg/mL) clear to slightly opalescent, colorless to light yellow solution in a single-dose vial.
153 mg/1.7 mL (90 mg/mL) clear to slightly opalescent, colorless to light yellow solution in a single-dose vial.

### 4 Contraindications

### None.

### 5 Warnings and Precautions

### 5.1 Cytokine Release Syndrome

TECVAYLI can cause cytokine release syndrome (CRS), including life-threatening or fatal reactions [see Adverse Reactions (6.1)].
In the clinical trial, CRS occurred in 72% of patients who received TECVAYLI at the recommended dose, with Grade 1 CRS occurring in 50% of patients, Grade 2 in 21%, and Grade 3 in 0.6%. Recurrent CRS occurred in 33% of patients. Most patients experienced CRS following step-up dose 1 (42%), step-up dose 2 (35%), or the initial treatment dose (24%). Less than 3% of patients developed first occurrence of CRS following subsequent doses of TECVAYLI. The median time to onset of CRS was 2 (range: 1 to 6) days after the most recent dose with a median duration of 2 (range: 1 to 9) days.
Clinical signs and symptoms of CRS included, but were not limited to, fever, hypoxia, chills, hypotension, sinus tachycardia, headache, and elevated liver enzymes (aspartate aminotransferase and alanine aminotransferase elevation).
Initiate therapy according to TECVAYLI step-up dosing schedule to reduce risk of CRS [see Dosage and Administration (2.1, 2.4)]. Administer pretreatment medications to reduce risk of CRS and monitor patients following administration of TECVAYLI accordingly [see Dosage and Administration (2.2, 2.4)].
At the first sign of CRS, immediately evaluate patient for hospitalization. Administer supportive care based on severity and consider further management per current practice guidelines. Withhold or permanently discontinue TECVAYLI based on severity [see Dosage and Administration (2.4)].
TECVAYLI is available only through a restricted program under a REMS [see Warnings and Precautions (5.3)].

### 5.2 Neurologic Toxicity Including ICANS

TECVAYLI can cause serious or life-threatening neurologic toxicity, including Immune Effector Cell-Associated Neurotoxicity Syndrome (ICANS) [see Adverse Reactions (6.1)].
In the clinical trial, neurologic toxicity occurred in 57% of patients who received TECVAYLI at the recommended dose, with Grade 3 or 4 neurologic toxicity occurring in 2.4% of patients. The most frequent neurologic toxicities were headache (25%), motor dysfunction (16%), sensory neuropathy (15%), and encephalopathy (13%).
With longer follow-up, Grade 4 seizure and fatal Guillain-Barré syndrome (one patient each) occurred in patients who received TECVAYLI.
In the clinical trial, ICANS was reported in 6% of patients who received TECVAYLI at the recommended dose [see Adverse Reactions (6.1)]. Recurrent ICANS occurred in 1.8% of patients. Most patients experienced ICANS following step-up dose 1 (1.2%), step-up dose 2 (0.6%), or the initial treatment dose (1.8%). Less than 3% of patients developed first occurrence of ICANS following subsequent doses of TECVAYLI. The median time to onset of ICANS was 4 (range: 2 to 8) days after the most recent dose with a median duration of 3 (range: 1 to 20) days. The most frequent clinical manifestations of ICANS reported were confusional state and dysgraphia. The onset of ICANS can be concurrent with CRS, following resolution of CRS, or in the absence of CRS.
Monitor patients for signs and symptoms of neurologic toxicity during treatment. At the first sign of neurologic toxicity, including ICANS, immediately evaluate patient and provide supportive therapy based on severity. Withhold or permanently discontinue TECVAYLI based on severity per recommendations and consider further management per current practice guidelines [see Dosage and Administration (2.4)].
Due to the potential for neurologic toxicity, patients receiving TECVAYLI are at risk of depressed level of consciousness [see Adverse Reactions (6.1)]. Advise patients to refrain from driving or operating heavy or potentially dangerous machinery during and for 48 hours after completion of TECVAYLI step-up dosing schedule and in the event of new onset of any neurologic toxicity symptoms until neurologic toxicity resolves [see Dosage and Administration (2.1)].
TECVAYLI is available only through a restricted program under a REMS [see Warnings and Precautions (5.3)].

### 5.3 TECVAYLI REMS

TECVAYLI is available only through a restricted program under a REMS called the TECVAYLI REMS because of the risks of CRS and neurologic toxicity, including ICANS [see Warnings and Precautions (5.1, 5.2)].
Notable requirements of the TECVAYLI REMS include the following:
Prescribers must be certified with the program by enrolling and completing training.
Prescribers must counsel patients receiving TECVAYLI about the risk of CRS and neurologic toxicity, including ICANS, and provide patients with Patient Wallet Card.
Pharmacies and healthcare settings that dispense TECVAYLI must be certified with the TECVAYLI REMS program and must verify prescribers are certified through the TECVAYLI REMS program.
Wholesalers and distributers must only distribute TECVAYLI to certified pharmacies or healthcare settings.
   Further information about the TECVAYLI REMS program is available at www.TECVAYLIREMS.com or by telephone at 1-855-810-8064.

### 5.4 Hepatotoxicity

TECVAYLI can cause hepatotoxicity, including fatalities. In patients who received TECVAYLI at the recommended dose in the clinical trial, there was one fatal case of hepatic failure. Elevated aspartate aminotransferase (AST) occurred in 34% of patients, with Grade 3 or 4 elevations in 1.2%. Elevated alanine aminotransferase (ALT) occurred in 28% of patients, with Grade 3 or 4 elevations in 1.8%. Elevated total bilirubin occurred in 6% of patients with Grade 3 or 4 elevations in 0.6%. Liver enzyme elevation can occur with or without concurrent CRS.
Monitor liver enzymes and bilirubin at baseline and during treatment as clinically indicated. Withhold TECVAYLI or consider permanent discontinuation of TECVAYLI based on severity [see Dosage and Administration (2.4)].

### 5.5 Infections

TECVAYLI can cause severe, life-threatening, or fatal infections. In patients who received TECVAYLI at the recommended dose in the clinical trial, serious infections, including opportunistic infections, occurred in 30% of patients, with Grade 3 or 4 infections in 35%, and fatal infections in 4.2% [see Adverse Reactions (6.1)].
Monitor patients for signs and symptoms of infection prior to and during treatment with TECVAYLI and treat appropriately. Administer prophylactic antimicrobials according to guidelines [see Dosage and Administration (2.2)].
Withhold TECVAYLI or consider permanent discontinuation of TECVAYLI based on severity [see Dosage and Administration (2.4)].
Monitor immunoglobulin levels during treatment with TECVAYLI and treat according to guidelines, including infection precautions and antibiotic or antiviral prophylaxis [see Dosage and Administration (2.2)].

### 5.6 Neutropenia

TECVAYLI can cause neutropenia and febrile neutropenia. In patients who received TECVAYLI at the recommended dose in the clinical trial, decreased neutrophils occurred in 84% of patients, with Grade 3 or 4 decreased neutrophils in 56%. Febrile neutropenia occurred in 3% of patients [see Adverse Reactions (6.1)].
Monitor complete blood cell counts at baseline and periodically during treatment and provide supportive care per local institutional guidelines.
Monitor patients with neutropenia for signs of infection.
Withhold TECVAYLI based on severity [see Dosage and Administration (2.4)].

### 5.7 Hypersensitivity and Other Administration Reactions

TECVAYLI can cause both systemic administration-related reactions and local injection-site reactions.

### Systemic Reactions

In patients who received TECVAYLI at the recommended dose in the clinical trial, 1.2% of patients experienced systemic-administration reactions, which included Grade 1 recurrent pyrexia and Grade 1 swollen tongue.

### Local Reactions

In patients who received TECVAYLI at the recommended dose in the clinical trial, injection-site reactions occurred in 35% of patients with Grade 1 injection-site reactions in 30% and Grade 2 in 4.8%.
Withhold TECVAYLI or consider permanent discontinuation of TECVAYLI based on severity [see Dosage and Administration (2.4)].

### 5.8 Embryo-Fetal Toxicity

Based on its mechanism of action, TECVAYLI may cause fetal harm when administered to a pregnant woman. Advise pregnant women of the potential risk to the fetus. Advise females of reproductive potential to use effective contraception during treatment with TECVAYLI and for 5 months after the last dose [see Use in Specific Populations (8.1, 8.3)].

### 6 Adverse Reactions

The following adverse reactions are also described elsewhere in the labeling:
Cytokine Release Syndrome [see Warnings and Precautions (5.1)]
Neurologic Toxicity including ICANS [see Warnings and Precautions (5.2)]
Hepatotoxicity [see Warnings and Precautions (5.4)]
Infections [see Warnings and Precautions (5.5)]
Neutropenia [see Warnings and Precautions (5.6)]
Hypersensitivity and Other Administration Reactions [see Warnings and Precautions (5.7)]

### 6.1 Clinical Trials Experience

Because clinical trials are conducted under widely varying conditions, adverse reaction rates observed in the clinical trials of a drug cannot be directly compared to rates in the clinical trials of another drug and may not reflect the rates observed in practice.

### Relapsed/Refractory Multiple Myeloma

### MajesTEC-1

The safety of TECVAYLI was evaluated in MajesTEC-1 [see Clinical Studies (14)] which included adult patients with relapsed or refractory multiple myeloma. Patients received step-up doses of 0.06 mg/kg and 0.3 mg/kg of TECVAYLI followed by TECVAYLI 1.5 mg/kg, subcutaneously once weekly (N=165). Among patients who received TECVAYLI, 47% were exposed for 6 months or longer and 7% were exposed for one year or longer.
The median age of patients who received TECVAYLI was 64 years (range: 33 to 84 years); 58% were male; 81% were White, 13% were Black or African American, and 2% were Asian.
Serious adverse reactions occurred in 54% of patients who received TECVAYLI. Serious adverse reactions in >2% of patients included pneumonia (15%), cytokine release syndrome (8%), sepsis (6%), general physical health deterioration (6%), COVID-19 (6%), acute kidney injury (4.8%), pyrexia (4.8%), musculoskeletal pain (2.4%), and encephalopathy (2.4%).
Fatal adverse reactions occurred in 5% of patients who received TECVAYLI, including COVID-19 (1.8%), pneumonia (1.8%), septic shock (0.6%), acute renal failure (0.6%), and hemoperitoneum (0.6%).
Permanent discontinuation of TECVAYLI due to adverse reactions occurred in 1.2% of patients. Adverse reactions resulting in permanent discontinuation of TECVAYLI included pneumonia (adenoviral and pneumocystis jirovecii pneumonia in the same patient) and hypercalcemia.
Dosage interruptions of TECVAYLI due to an adverse reaction occurred in 73% of patients. Adverse reactions which required dosage interruption in >5% of patients included neutropenia, pneumonia, pyrexia, cytokine release syndrome, upper respiratory tract infection, and COVID-19.
The most common adverse reactions (>20%) were pyrexia, CRS, musculoskeletal pain, injection site reaction, fatigue, upper respiratory tract infection, nausea, headache, pneumonia, and diarrhea. The most common Grade 3 to 4 laboratory abnormalities (>20%) were decreased lymphocytes, decreased neutrophils, decreased white blood cells, decreased hemoglobin, and decreased platelets.
Table 10 summarizes the adverse reactions in MajesTEC-1.

TABLE 10
Adverse Reactions (≥10%) in Patients with Multiple
Myeloma Who Received TECVAYLI in MajesTEC-1

TECVAYLI
(N = 165)

| | Any Grade | Grade 3 or 4 |
|---|---|---|
| Adverse Reactions | (%) | (%) |
| General disorders and | | |
| administration site conditions | | |
| Pyrexia | 76 | 3^{#} |
| Injection site reaction¹ | 37 | 0.6^{#} |
| Fatigue² | 33 | 2.4^{#} |
| Chills | 16 | 0 |
| Pain³ | 15 | 1.8^{#} |
| Edema⁴ | 13 | 0 |
| Immune system disorders | | |
| Cytokine release syndrome | 72 | 0.6^{#} |
| Hypogammaglobulinemia⁵ | 11 | 1.2^{#} |
| Musculoskeletal and | | |
| connective tissue disorders | | |
| Musculoskeletal pain⁶ | 44 | 4.2^{#} |
| Bone pain | 16 | 3^{#} |
| Infections | | |
| Upper respiratory tract infection⁷ | 26 | 2.4^{#} |
| Pneumonia⁸* | 24 | 15 |
| Urinary tract infection⁹ | 11 | 5^{#} |
| Gastrointestinal disorders | | |
| Nausea | 25 | 0.6^{#} |
| Diarrhea | 21 | 2.4^{#} |
| Constipation | 18 | 0 |
| Vomiting | 12 | 0.6^{#} |
| Nervous system disorders | | |
| Headache | 25 | 0.6^{#} |
| Motor dysfunction¹⁰ | 16 | 0 |
| Sensory neuropathy¹¹ | 15 | 1.2^{#} |
| Encephalopathy¹² | 13 | 0 |
| Vascular disorders | | |
| Hypotension | 18 | 1.2^{#} |
| Hemorrhage¹³* | 12 | 1.8 |
| Hypertension¹⁴ | 12 | 4.8^{#} |
| Respiratory, thoracic, | | |
| and mediastinal disorders | | |
| Hypoxia | 18 | 1.8 |
| Cough¹⁵ | 15 | 0 |
| Cardiac disorders | | |
| Cardiac arrhythmia¹⁶ | 16 | 1.8 |
| Metabolism and nutrition disorders | | |
| Decreased appetite | 11 | 0.6^{#} |
| Renal and urinary disorders | | |
| Acute kidney injury¹⁷ | 11 | 3.6 |

Adverse reactions were graded based on CTCAE Version 4.03, with the exception of CRS, which was graded per ASTCT 2019 criteria.
¹Injection site reaction includes application site erythema, injection site bruising, injection site cellulitis, injection site discomfort, injection site erythema, injection site hematoma, injection site induration, injection site inflammation, injection site edema, injection site pruritus, injection site rash, injection site reaction and injection site swelling.
²Fatigue includes asthenia and fatigue.
³Pain includes ear pain, flank pain, groin pain, oropharyngeal pain, pain, pain in jaw, toothache and tumor pain.
⁴Edema includes face edema, fluid overload, fluid retention, edema peripheral and peripheral swelling.
⁵Hypogammaglobulinemia includes hypogammaglobulinemia and hypoglobulinemia.
⁶Musculoskeletal pain includes arthralgia, back pain, muscle discomfort, musculoskeletal chest pain, musculoskeletal pain, myalgia, neck pain, non-cardiac chest pain and pain in extremity.
⁷Upper respiratory tract infection includes bronchitis, influenza like illness, nasopharyngitis, pharyngitis, respiratory tract infection, respiratory tract infection bacterial, rhinitis, rhinovirus infection, sinusitis, tracheitis, upper respiratory tract infection and viral upper respiratory tract infection.
⁸Pneumonia includes COVID-19 pneumonia, enterobacter pneumonia, lower respiratory tract infection, metapneumovirus pneumonia, pneumocystis jirovecii pneumonia, pneumonia, pneumonia adenoviral, pneumonia klebsiella, pneumonia moraxella, pneumonia pneumococcal, pneumonia pseudomonal, pneumonia respiratory syncytial viral, pneumonia staphylococcal and pneumonia viral.
⁹Urinary tract infection includes cystitis, cystitis escherichia, cystitis klebsiella, escherichia urinary tract infection, urinary tract infection and urinary tract infection bacterial.
¹⁰Motor dysfunction includes cogwheel rigidity, dysgraphia, dysphonia, gait disturbance, hypokinesia, muscle rigidity, muscle spasms, muscular weakness, peroneal nerve palsy, psychomotor hyperactivity, tremor and VI^{th} nerve paralysis.
¹¹Sensory neuropathy includes dysesthesia, hypoesthesia, hypoesthesia oral, neuralgia, paresthesia, paresthesia oral, peripheral sensory neuropathy, sciatica and vestibular neuronitis.
¹²Encephalopathy includes agitation, apathy, aphasia, confusional state, delirium, depressed level of consciousness, disorientation, dyscalculia, hallucination, lethargy, memory impairment, mental status changes and somnolence.
¹³Hemorrhage includes conjunctival hemorrhage, epistaxis, hematoma, hematuria, hemoperitoneum, hemorrhoidal hemorrhage, lower gastrointestinal hemorrhage, melena, mouth hemorrhage and subdural hematoma.
¹⁴Hypertension includes essential hypertension and hypertension.
¹⁵Cough includes allergic cough, cough, productive cough and upper-airway cough syndrome.
¹⁶Cardiac arrhythmia includes atrial flutter, cardiac arrest, sinus bradycardia, sinus tachycardia, supraventricular tachycardia, tachycardia and ventricular tachycardia.
¹⁷Acute kidney injury includes acute kidney injury and renal impairment.
^{#}Only grade 3 adverse reactions occurred.
*Includes the following fatal adverse reactions: hemorrhage (n = 1), pneumonia (n = 3).

Clinically relevant adverse reactions in <10% of patients who received TECVAYLI included febrile neutropenia, sepsis, ICANS, seizure, Guillain-Barré syndrome, hepatic failure, and new onset or reactivated viral infections (including adenovirus, hepatitis B virus (HBV), cytomegalovirus (CMV), varicella zoster virus (VZV), and herpes simplex virus (HSV)).
Table 11 summarizes laboratory abnormalities in MajesTEC-1.

TABLE 11
Select Laboratory Abnormalities (≥30%) That
Worsened from Baseline in Patients with Multiple
Myeloma Who Received TECVAYLI in MajesTEC-1

TECVAYLI
(N = 165¹)

| Laboratory Abnormality | All Grades (%) | Grade 3 or 4 (%) |
|---|---|---|
| Hematology | | |
| Lymphocyte count | 92 | 84 |
| decreased | | |
| White blood cell | 86 | 41 |
| decreased | | |
| Neutrophil count | 84 | 56 |
| decreased | | |
| Platelet count decreased | 71 | 22 |
| Hemoglobin decreased | 67 | 33 |
| Chemistry | | |
| Albumin decreased | 68 | 6 |
| Alkaline phosphatase | 42 | 2.4 |
| increased | | |
| Phosphorus decreased | 38 | 13 |
| Gamma-glutamyl | 37 | 8 |
| transferase increased | | |
| Sodium decreased | 35 | 10 |
| Aspartate | 34 | 1.2 |
| aminotransferase | | |
| increased | | |
| Calcium (corrected) | 31 | 1.2 |
| decreased | | |
| Creatinine increased | 30 | 3 |

¹The denominator used to calculate the rate varied from 164 to 165 based on the number of patients with a baseline value and at least one post-treatment value.
Laboratory toxicity grades are derived based on the NCI CTCAE (National Cancer Institute Common Terminology Criteria for Adverse Events) Version 4.03.

### 7 Drug Interactions

TECVAYLI causes release of cytokines [see Clinical Pharmacology (12.2)] that may suppress activity of cytochrome P450 (CYP) enzymes, resulting in increased exposure of CYP substrates. The highest risk of drug-drug interaction is expected to occur from initiation of TECVAYLI step-up dosing schedule up to 7 days after the first treatment dose and during and after CRS [see Warnings and Precautions (5.1)]. Monitor for toxicity or concentrations of drugs that are CYP substrates where minimal concentration changes may lead to serious adverse reactions. Adjust the dose of the concomitant CYP substrate drug as needed.

### 8 Use in Specific Populations

### 8.1 Pregnancy

### Risk Summary

Based on the mechanism of action, TECVAYLI may cause fetal harm when administered to a pregnant woman [see Clinical Pharmacology (12.1)]. There are no available data on the use of TECVAYLI in pregnant women. No animal reproductive or developmental toxicity studies have been conducted with TECVAYLI. Teclistamab-cqyv causes T-cell activation and cytokine release; immune activation may compromise pregnancy maintenance. Human immunoglobulin G (IgG) is known to cross the placenta; therefore, teclistamab-cqyv has the potential to be transmitted from the mother to the developing fetus. Advise women of the potential risk to the fetus.
TECVAYLI is associated with hypogammaglobulinemia, therefore, assessment of immunoglobulin levels in newborns of mothers treated with TECVAYLI should be considered.
In the U.S. general population, the estimated background risk of major birth defects and miscarriage in clinically recognized pregnancies is 2% to 4% and 15% to 20%, respectively.

### 8.2 Lactation

### Risk Summary

There are no data on the presence of teclistamab-cqyv in human milk, the effect on the breastfed child, or the effects on milk production. Maternal IgG is known to be present in human milk. The effects of local gastrointestinal exposure and limited systemic exposure in the breastfed child to TECVAYLI are unknown. Because of the potential for serious adverse reactions in a breastfed child, advise patients not to breastfeed during treatment with TECVAYLI and for 5 months after the last dose.

### 8.3 Females and Males of Reproductive Potential

TECVAYLI may cause fetal harm when administered to a pregnant woman [see Use in Specific Populations (8.1)].

### Pregnancy Testing

Verify pregnancy status of females of reproductive potential prior to initiating TECVAYLI.

### Contraception

### Females

Advise females of reproductive potential to use effective contraception during treatment and for 5 months after the last dose of TECVAYLI.

### 8.4 Pediatric Use

The safety and efficacy of TECVAYLI have not been established in pediatric patients.

### 8.5 Geriatric Use

Of the 165 patients with relapsed or refractory multiple myeloma treated with TECVAYLI in MajesTEC-1 at the recommended dosage, 48% were 65 years of age or older, and 15% were 75 years of age or older. No overall differences in safety or effectiveness were observed between patients 65 to 74 years of age compared to younger patients. There is an insufficient number of patients 75 years of age or older to assess whether there are differences in safety or effectiveness.

### 11 Description

Teclistamab-cqyv, a bispecific B-cell maturation antigen (BCMA)-directed CD3 T-cell engager, is a humanized immunoglobulin G4-proline, alanine, alanine (IgG4-PAA) antibody. Teclistamab-cqyv is produced in Chinese Hamster Ovary (CHO) cells using recombinant DNA technology. Teclistamab-cqyv consists of an anti-BCMA heavy chain and light chain and an anti-CD3 heavy chain and light chain with two interchain disulfide bonds connecting the two arms. The molecular weight of teclistamab-cqyv is approximately 146 kDa.

TECVAYLI™ (teclistamab-cqyv) injection is a sterile, preservative-free, clear to slightly opalescent, colorless to light yellow solution supplied in a single-dose vial for subcutaneous administration.
Each TECVAYLI 3 mL single-dose vial contains 30 mg of teclistamab-cqyv, edetate disodium (0.054 mg), glacial acetic acid (0.72 mg), polysorbate 20 (1.2 mg), sodium acetate (2.7 mg), sucrose (240 mg), and Water for Injection, USP.
Each TECVAYLI 1.7 mL single-dose vial contains 153 mg of teclistamab-cqyv, edetate disodium (0.031 mg), glacial acetic acid (0.41 mg), polysorbate 20 (0.68 mg), sodium acetate (1.5 mg), sucrose (140 mg), and Water for Injection, USP.

### 12 Clinical Pharmacology

### 12.1 Mechanism of Action

Teclistamab-cqyv is a bispecific T-cell engaging antibody that binds to the CD3 receptor expressed on the surface of T-cells and B-cell maturation antigen (BCMA) expressed on the surface of multiple myeloma cells and some healthy B-lineage cells.
In vitro, teclistamab-cqyv activated T-cells, caused the release of various proinflammatory cytokines, and resulted in the lysis of multiple myeloma cells.

### 12.2 Pharmacodynamics

Serum concentrations of cytokines (IL-6, IL-10, TNF-α, and IFN-γ) and IL-2R were measured before and after administration of step-up dose 1, step-up dose 2, and the first three treatment doses of TECVAYLI. Increased concentrations of IL-6, IL-10, and IL-2R were observed during this period.

### 12.3 Pharmacokinetics

The Cₘₐₓ and AUCₜₐᵤ of teclistamab-cqyv after the first subcutaneous treatment dose increase proportionally over a dosage range of 0.08 mg/kg to 3 mg/kg (0.05 to 2 times the approved recommended treatment dosage). Ninety percent of steady state exposure was achieved after 12 weekly treatment doses. The mean accumulation ratio between the first and 13^{th} weekly treatment dose of teclistamab-cqyv 1.5 mg/kg was 4.2-fold for Cₘₐₓ, 4.1-fold for C_{trough}, and 5.3-fold for AUCₜₐᵤ.
The Cₘₐₓ, C_{trough}, and AUCₜₐᵤ of teclistamab-cqyv are presented in Table 12.

TABLE 12
Pharmacokinetic Parameters of Teclistamab-cqyv
for the 13^{th} Treatment Dose of 1.5 mg/kg^{a}

| Pharmacokinetic | Teclistamab-cqyv | |
|---|---|---|
| Parameter | Geometric Mean (CV %) | |
| Cₘₐₓ (mcg/mL) | 23.8 | (55%) |
| C_{trough} (mcg/mL) | 21.1 | (63%) |
| AUCₜₐᵤ (mcg · h/mL) | 3,838 | (57%) |

SD = standard deviation; Cₘₐₓ = Maximum serum teclistamab-cqyv concentration; C_{trough} = Serum teclistamab-cqyv concentration prior to next dose; CV = geometric coefficient of variation; AUCₜₐᵤ = Area under the concentration-time curve over the weekly dosing interval.
^{a}Following administration of teclistamab-cqyv in patients with relapsed or refractory multiple myeloma (MajesTEC-1).

### Absorption

The mean bioavailability of teclistamab-cqyv was 72% when administered subcutaneously. The median (range) Tₘₐₓ of teclistamab-cqyv after the first and 13^{th} treatment doses were 139 (19 to 168) hours and 72 (24 to 168) hours, respectively.

### Distribution

The mean (coefficient of variation [CV]%) volume of distribution of teclistamab-cqyv was 5.63 L (29%).

### Elimination

Teclistamab-cqyv clearance decreases over time, with a mean (CV %) maximal reduction from baseline to the 13^{th} treatment dose of 40.8% (56%). The geometric mean (CV %) clearance is 0.472 L/day (64%) at the 13^{th} treatment dose. Patients who discontinue teclistamab-cqyv after the 13^{th} treatment dose are expected to have a 50% reduction from Cₘₐₓ in teclistamab-cqyv concentration at a median (5^{th} to 95^{th} percentile) time of 15 (7 to 33) days after Tₘₐₓ and a 97% reduction from Cₘₐₓ in teclistamab-cqyv concentration at a median time of 69 (32 to 163) days after Tₘₐₓ.

### Specific Populations

The volume of distribution and clearance of teclistamab-cqyv increase with increasing body weight (41 kg to 139 kg).
There were no clinically significant differences in the pharmacokinetics of teclistamab-cqyv based on age (24 to 84 years), sex, race (White, Black or African American), ethnicity (Hispanic/Latino, not Hispanic/Latino), mild or moderate renal impairment (estimated glomerular filtration rate [eGFR] by Modification of Diet in Renal Disease [MDRD] method: 30 to 89 mL/min), or mild hepatic impairment (total bilirubin less than or equal to upper limit of normal [ULN] with AST greater than ULN or total bilirubin greater than 1 to 1.5 times ULN with any AST). The effects of severe renal impairment (eGFR less than 30 mL/min) or moderate to severe hepatic impairment (total bilirubin greater than 1.5 times ULN with any AST) on the pharmacokinetics of teclistamab-cqyv are unknown.

### Drug Interaction Studies

No clinical studies evaluating the drug interaction potential of teclistamab-cqyv have been conducted.

### 12.6 Immunogenicity

The observed incidence of anti-drug antibodies is highly dependent on the sensitivity and specificity of the assay. Differences in assay methods preclude meaningful comparisons of the incidence of anti-drug antibodies in the studies described below with the incidence of anti-drug antibodies in other studies, including those of teclistamab-cqyv or of other teclistamab products.
During treatment in MajesTEC-1 (up to 27 months), 1/186 (0.5%) of patients treated with subcutaneous TECVAYLI at various dosages developed anti-teclistamab-cqyv antibodies. Because of the low occurrence of anti-drug antibodies, the effect of these antibodies on the pharmacokinetics, pharmacodynamics, safety, and/or effectiveness of teclistamab products is unknown.

### 13 Nonclinical Toxicology

### 13.1 Carcinogenesis, Mutagenesis, Impairment of Fertility

No carcinogenicity or genotoxicity studies have been conducted with teclistamab-cqyv.
No studies have been conducted to evaluate the effects of teclistamab-cqyv on fertility.

### 14 Clinical Studies

The efficacy of TECVAYLI was evaluated in patients with relapsed or refractory multiple myeloma in a single-arm, open-label, multi-center study (MajesTEC-1, NCT03145181 [Phase 1] and NCT04557098 [Phase 2]). The study included patients who had previously received at least three prior therapies, including a proteasome inhibitor, an immunomodulatory agent, and an anti-CD38 monoclonal antibody. The study excluded patients who had stroke, seizure, allogeneic stem cell transplantation within the past 6 months, Eastern Cooperative Oncology Group (ECOG) performance score of 2 or higher, known active CNS involvement or clinical signs of meningeal involvement of multiple myeloma, or active or documented history of autoimmune disease, with the exception of vitiligo, Type 1 diabetes, and prior autoimmune thyroiditis.
Patients received step-up doses of 0.06 mg/kg and 0.3 mg/kg of TECVAYLI followed by TECVAYLI 1.5 mg/kg subcutaneously once weekly thereafter until disease progression or unacceptable toxicity [see Dosage and Administration (2.1)].
The efficacy population included 110 patients. The median age was 66 (range: 33 to 82) years with 16% of patients 75 years of age or older; 56% were male; 91% were White, 5% were Black or African American, 3% were Asian. The International Staging System (ISS) at study entry was Stage I in 50%, Stage II in 38%, and Stage III in 12% of patients. High-risk cytogenetics (presence of del(17p), t(4; 14) and t(14; 16)) were present in 25% of patients. Seventeen percent of patients had extramedullary plasmacytomas. Patients with prior BCMA-targeted therapy were not included in the efficacy population.
The median number of prior lines of therapy was 5 (range: 2 to 14); 78% of patients had received at least 4 prior lines of therapy. Eighty-one percent of patients received prior stem cell transplantation. All patients had received prior therapy with a proteasome inhibitor, an immunomodulatory agent and an anti-CD38 monoclonal antibody, and 76% were triple-class refractory (refractory to a proteasome inhibitor, an immunomodulatory agent and an anti-CD38 monoclonal antibody).
Efficacy was established based on overall response rate (ORR) as determined by the Independent Review Committee (IRC) assessment using International Myeloma Working Group (IMWG) 2016 criteria (see Table 13).
The median time to first response was 1.2 months (range: 0.2 to 5.5 months). With a median follow-up of 7.4 months among responders, the estimated duration of response (DOR) rate was 90.6% (95% CI: 80.3%, 95.7%) at 6 months and 66.5% (95% CI: 38.8%, 83.9%) at 9 months.

TABLE 13
Efficacy Results for MajesTEC-1

| | N = 110 | |
|---|---|---|
| Overall response rate (ORR: | 68 | (61.8) |
| SCR + CR + VGPR + PR) n(%) | | |
| 95% CI (%) | (52.1, 70.9) | |
| Complete response (CR) or better^{a} | 31 | (28.2) |
| Very good partial response (VGPR) | 32 | (29.1) |
| Partial response (PR) | 5 | (4.5) |

Duration of Response (DOR) (months)

| | |
|---|---|
| DOR (Months): Median (95% CI) | NE (9.0, NE) |

NE = not estimable
^{a}Complete response or better = Stringent complete response (sCR) + complete response (CR).

### 16 how Supplied/Storage and Handling

TECVAYLI™ (teclistamab-cqyv) injection is a sterile, preservative-free, clear to slightly opalescent, colorless to light yellow solution supplied as follows:
One 30 mg/3 mL (10 mg/mL) single-dose vial in a carton: NDC: 57894-449-01
One 153 mg/1.7 mL (90 mg/mL) single-dose vial in a carton: NDC: 57894-450-01
   Store refrigerated at 2° C. to 8° C. (36° F. to 46° F.) in the original carton to protect from light.
   Do not freeze.

### 17 Patient Counseling Information

Advise the patient to read the FDA-approved patient labeling (Medication Guide).

### Cytokine Release Syndrome (CRS)

Discuss the signs and symptoms associated with CRS, including fever, hypoxia, chills, hypotension, sinus tachycardia, headache, and elevated liver enzymes. Advise patients to immediately contact their healthcare provider if they experience any signs or symptoms of CRS. Advise patients that they will be hospitalized for 48 hours after administration of all doses within the TECVAYLI step-up dosing schedule [see Dosage and Administration (2.4) and Warnings and Precautions (5.1)].

### Neurologic Toxicity Including ICANS

Discuss the signs and symptoms associated with neurologic toxicity, including ICANS, including headache, confusion, dysgraphia, motor dysfunction, neuropathy, or encephalopathy. Advise patients to immediately contact their healthcare provider if they experience any signs or symptoms of neurologic toxicity. Advise patients to refrain from driving or operating heavy or potentially dangerous machinery during and for 48 hours after completion of TECVAYLI step-up dosing schedule and in the event of new onset of any neurologic toxicity symptoms until neurologic toxicity resolves [see Dosage and Administration (2.4) and Warnings and Precautions (5.2)].

### TECVAYLI REMS

TECVAYLI is available only through a restricted program called TECVAYLI REMS. Inform patients that they will be given a TECVAYLI Patient Wallet Card that they should carry with them at all times and show to all of their healthcare providers. This card describes signs and symptoms of CRS and neurologic toxicity which, if experienced, should prompt the patient to immediately seek medical attention [see Warnings and Precautions (5.3)].

### Hepatotoxicity

Advise patients that liver enzyme elevations may occur and that they should report symptoms that may indicate liver toxicity, including fatigue, anorexia, right upper abdominal discomfort, dark urine, or jaundice [see Warnings and Precautions (5.4)].

### Infections

Discuss the signs and symptoms of infection [see Dosage and Administration (2.4) and Warnings and Precautions (5.5)].

### Neutropenia

Discuss the signs and symptoms associated with neutropenia and febrile neutropenia [see Dosage and Administration (2.4) and Warnings and Precautions (5.6)].

### Hypersensitivity and Other Administration Reactions

Advise patients to immediately seek medical attention for any signs and symptoms of systemic administration-related reactions. Advise patients that local injection-site reactions may occur and to report any severe reactions [see Warnings and Precautions (5.7)].

### Embryo-Fetal Toxicity

Advise pregnant women to inform their healthcare provider if they are pregnant or become pregnant. Advise females of reproductive potential of the potential risk to a fetus and to use effective contraception during treatment with TECVAYLI and for 5 months after the last dose [see Warnings and Precautions (5.8) and Use in Specific Populations (8.1, 8.3)].

### Lactation

Advise women not to breastfeed during treatment with TECVAYLI and for 5 months after the last dose [see Use in Specific Populations (8.2)].

### Manufactured by:

### Janssen Biotech, Inc.

### Horsham, PA 19044

### U.S. License Number 1864

### © 2022 Janssen Pharmaceutical Companies

For patent information: www.janssenpatents.com

### Medication Guide

### TECVAYLI™ [tek vay′ lee]

### (Teclistamab-Cqyv) Injection, for Subcutaneous Use

What is the most important information I should know about TECVAYLI?
TECVAYLI may cause side effects that are serious, life-threatening or lead to death, including Cytokine Release Syndrome (CRS) and Neurologic problems.
Call your healthcare provider right away if you develop any of the signs or symptoms of CRS or neurologic problems listed below at any time during your treatment with TECVAYLI:
Cytokine Release Syndrome (CRS). Signs and symptoms of CRS may include:

| | |
|---|---|
| fever (100.4° F. or higher) | feeling anxious |
| difficulty breathing | confusion or restlessness |
| chills | headache |
| dizziness or lightheadedness | increased liver enzymes in your |
| | blood |
| fast heartbeat | |

Neurologic problems. Symptoms of neurologic problems with TECVAYLI include:

| | | |
|---|---|---|
| headache | confusion | changes in your handwriting |
| jerking | trouble | problems walking |
| movements | speaking | |
| rigid muscles | muscle spasms | muscle weakness in your |
| feeling restless | tremor | body or face |
| numbness and | double vision | hearing loss |
| tingling (feeling like | | burning, throbbing, or |
| “pins and needles”) | | stabbing pain |

Due to the risk of CRS and neurologic symptoms, you should be hospitalized for 48 hours after all doses of TECVAYLI that are part of the “step-up dosing schedule.” The “step-up dosing schedule” is when you receive the first 2 doses of TECVAYLI, which are called “step-up” doses, and then you receive the first “treatment dose” of TECVAYLI. After “step-up” dose 1 of TECVAYLI, the dose of TECVAYLI is increased. After “step-up” dose 2, the dose is increased again when you receive the first “treatment dose” of TECVAYLI.
   “Step-up dose 1” is given on day 1 of treatment. “Step-up dose 2” is usually given on day 4 of treatment. The first “treatment dose” is usually given on day 7 of treatment.
   Your healthcare provider will decide when you will receive “step-up dose 2” and your first “treatment dose.”
      “Step-up” dose 2 may be given between 2 to 4 days after “step-up” dose 1, or up to 7 days after “step-up” dose 1 if you have certain side effects with TECVAYLI.
      Your first “treatment dose” may be given between 2 to 4 days after “step-up” dose 2, or up to 7 days after “step-up” dose 2 if you have certain side effects with TECVAYLI.
   Your healthcare provider will decide the number of days to wait between your doses of TECVAYLI as well as how many treatments you will receive.
   If your dose of TECVAYLI is delayed for any reason, you may need to repeat the “step-up dosing schedule” to receive TECVAYLI.
   Before each “step-up” dose and your first “treatment dose” of TECVAYLI you will receive medicines to help reduce your risk of CRS. Your healthcare provider will decide if you need to receive medicines to help reduce your risk of CRS with future doses.
Your healthcare provider will monitor you for signs and symptoms of CRS and neurologic problems during treatment with TECVAYLI, as well as other side effects and treat you as needed.
Do not drive or operate heavy or dangerous machinery during and for 48 hours after your TECVAYLI “step-up dosing schedule” is completed, or at any time during treatment with TECVAYLI if you develop new neurologic symptoms until the symptoms go away.
   TECVAYLI is available only through the TECVAYLI Risk Evaluation and Mitigation Strategy (REMS) due to the risk of CRS and neurologic problems.
   You will receive a TECVAYLI Patient Wallet Card from your healthcare provider. Carry the TECVAYLI Patient Wallet Card with you at all times and show it to all of your healthcare providers. The TECVAYLI Patient Wallet Card lists signs and symptoms of CRS and neurologic problems.
   Get medical help right away if you develop any of the signs and symptoms listed on the TECVAYLI Patient Wallet Card. You may need to be treated in a hospital.
If you have any questions about TECVAYLI, ask your healthcare provider.
Your healthcare provider may temporarily stop or completely stop your treatment with TECVAYLI if you develop CRS, neurologic problems or any other side effects that are severe.
   See “What are the possible side effects of TECVAYLI?” for more information about side effects.

### What is TECVAYLI?

TECVAYLI is a prescription medicine to treat adults with multiple myeloma who:
have already received at least 4 treatment regimens, including a proteasome inhibitor, an immunomodulatory agent and an anti-CD38 monoclonal antibody to treat their multiple myeloma, and
their cancer has come back or did not respond to prior treatment
   It is not known if TECVAYLI is safe and effective in children.
   Before you receive TECVAYLI, tell your healthcare provider about all of your medical conditions, including if you:
have an infection
are pregnant or plan to become pregnant. TECVAYLI may harm your unborn baby.
   Your healthcare provider should do a pregnancy test before you start treatment with TECVAYLI.
   You should use effective birth control (contraception) during treatment and for 5 months after your last dose of TECVAYLI.
   Tell your healthcare provider right away if you become pregnant or think that you may be pregnant during treatment with TECVAYLI.
are breastfeeding or plan to breastfeed. It is not known if TECVAYLI passes into your breast milk. Do not breastfeed during treatment and for 5 months after your last dose of TECVAYLI.
   Tell your healthcare provider about all the medicines you take, including prescription and over-the-counter medicines, vitamins, and herbal supplements.

### How Will I Receive TECVAYLI?

TECVAYLI will be given to you by your healthcare provider as an injection under your skin (subcutaneous injection), usually in your stomach-area (abdomen), your thigh or another area of your body may be injected.
See “What is the most important information I should know” at the beginning of this Medication Guide for information about how you will receive TECVAYLI.
If you miss any appointments, call your healthcare provider as soon as possible to reschedule your appointment. It is important for you to be monitored closely for side effects during treatment with TECVAYLI.

### What are the Possible Side Effects of TECVAYLI?

TECVAYLI may cause serious side effects, including:
See “What is the most important information I should know about TECVAYLI?”
Liver problems. TECVAYLI can cause liver problems that may lead to death. Increased bilirubin and liver enzymes in your blood are common with TECVAYLI and can also sometimes be severe These increases in liver enzymes can happen with or without you also having CRS. Your healthcare provider will monitor you for these problems before you start and during treatment with TECVAYLI. Tell your healthcare provider if you develop any symptoms of a liver problem including:

| | |
|---|---|
| tiredness | pain in your right upper stomach-area (abdomen) |
| loss of appetite | dark urine |
| | yellowing of your skin or white part of your eyes |

Infections. Upper respiratory tract infections and pneumonia are common with TECVAYLI. TECVAYLI can cause bacterial and viral infections that are severe, life-threatening, or that may lead to death.
   Your healthcare provider will monitor you for signs and symptoms of infection before and during treatment with TECVAYLI.
   Your healthcare provider may prescribe medicines for you to help prevent infections and treat you as needed if you develop an infection during treatment with TECVAYLI.
   Tell your healthcare provider right away if you get a fever, chills or any signs or symptoms of an infection.
Decreased white blood cell counts. Decreased white blood cell counts are common with TECVAYLI and can also be severe. Fever sometimes also happens with low white blood cell counts and may be a sign that you have an infection. Your healthcare provider will check your blood cell counts before you start and during treatment with TECVAYLI, and treat you as needed.
Allergic reactions and injection site reactions. TECVAYLI can cause allergic reactions that can affect your whole body (systemic), and also cause injection site reactions.
   Some people taking TECVAYLI can develop symptoms of an allergic reaction that can affect your whole body and may include fever or a swollen tongue. Get medical help right away if you develop symptoms of an allergic reaction during treatment with TECVAYLI.
   Injection site reactions are common with TECVAYLI and can include: redness, heat, swelling, bruising, bacterial skin infection (cellulitis), discomfort, blood collection under the skin at the injection site (hematoma), and rash. Tell your healthcare provider if you develop any severe injection site reactions.
      Your healthcare provider may temporarily or permanently stop TECVAYLI if you have any of the side effects listed above and they are severe.
      The most common side effects of TECVAYLI include:

| | |
|---|---|
| fever | upper respiratory tract infections and |
| pain in your joints and muscles, | pneumonia. See “Infections” above. |
| back and chest muscles, | nausea |
| and in your arms and legs | |
| tiredness and weakness | headache |
| | diarrhea |

The most common severe abnormal lab test results with TECVAYLI include: decreased white blood cells, red blood cells and platelets.
These are not all the possible side effects of TECVAYLI.
Call your doctor for medical advice about side effects. You may report side effects to FDA at 1-800-FDA-1088.
General Information about TECVAYLI.
Medicines are sometimes prescribed for purposes other than those listed in a Medication Guide.
You can ask your healthcare provider for information about TECVAYLI that is written for health professionals.

### What are the Ingredients of TECVAYLI?

Active ingredient: teclistamab-cqyv
Inactive ingredients: edetate disodium, glacial acetic acid, polysorbate 20, sodium acetate, sucrose, Water for Injection

### Manufactured by: Janssen Biotech, Inc., Horsham, PA 19044

### U.S. License Number 1864

### © 2022 Janssen Pharmaceutical Companies

For patent information: www.janssenpatents.com
For more information about TECVAYLI go to www.TECVAYLI.com or call 1-800-526-7736.
This Medication Guide has been approved by the U.S. Food and Drug Administration. Issued: October 2022

### Embodiments

The following list of embodiments is intended to complement, rather than displace or supersede, the previous descriptions.

Embodiment 1. An approved product comprising:
30 mg/3 mL (10 mg/mL) of teclistamab-cqyv in a single-dose vial; and/or
153 mg/1.7 mL (90 mg/mL) of teclistamab-cqyv in a single-dose vial.

Embodiment 2. The approved product of embodiment 1, comprising 30 mg/3 mL (10 mg/mL) of teclistamab-cqyv in a single-dose vial.

Embodiment 3. The approved product of embodiment 1, comprising 153 mg/1.7 mL (90 mg/mL) of teclistamab-cqyv in a single-dose vial.

Embodiment 4. The approved product of any one of the previous embodiments,
wherein the approved product is formulated for delivery to a human in a dosing schedule comprising:
   0.06 mg/kg administered as a first step-up dose on Day 1;
   0.3 mg/kg administered as a second step-up dose 2 to 7 days after the first step-up dose;
   1.5 mg/kg administered as a first treatment dose 2 to 7 days after the second step-up dose; and
   1.5 mg/kg administered as a subsequent treatment dose one week after the first treatment dose and once weekly thereafter.

Embodiment 5. The approved product of embodiment 4, wherein the second step-up dose is administered on Day 4.

Embodiment 6. The approved produce of embodiment 4 or 5, wherein the first treatment dose is administered on Day 7.

Embodiment 7. The approved product of any one of the previous embodiments,
wherein the approved product is formulated for delivery to a human in a dosing schedule comprising:
   0.06 mg/kg administered as a first step-up dose on Day 1;
   0.3 mg/kg administered as a second step-up dose on Day 4;
   1.5 mg/kg administered as a first treatment dose on Day 7; and
   1.5 mg/kg administered as a subsequent treatment dose one week after the first treatment dose and once weekly thereafter.

Embodiment 8. The approved product of any one of the previous embodiments, wherein the approved product is a biosimilar.

Embodiment 9. The approved product of any one of the previous embodiments, in a 3 ml single-dose vial comprising 30 mg of teclistamab-cqyv, edetate disodium, glacial acetic acid, polysorbate 20, sodium acetate, sucrose, and water for injection, USP.

Embodiment 10. The approved product of any one of the previous embodiments, in a 3 ml single-dose vial comprising 30 mg of teclistamab-cqyv, 0.054 mg edetate disodium, 0.72 mg glacial acetic acid, 1.2 mg polysorbate 20, 2.7 mg sodium acetate, 240 mg sucrose, and water for injection, USP.

Embodiment 11. The approved product of any one of embodiments 1-8, in a 1.7 ml single-dose vial comprising 153 mg of teclistamab-cqyv, edetate disodium, glacial acetic acid, polysorbate 20, sodium acetate, sucrose, and water for injection, USP.

Embodiment 12. The approved product of any one of embodiments 1-8, in a 1.7 ml single-dose vial comprising 153 mg of teclistamab-cqyv, 0.031 mg edetate disodium, 0.41 mg glacial acetic acid, 0.68 mg polysorbate 20, 1.5 mg sodium acetate, 140 mg sucrose, and water for injection, USP.

Embodiment 13. The approved product of any of previous embodiments, in combination with a corticosteroid, a histamine-1 (H1) receptor antagonist, and an antipyretic.

Embodiment 14. The approved product of embodiment 13, in combination with 16 mg of oral or intravenous dexamethasone, 50 mg or equivalent of oral or intravenous diphenhydramine, and 650 mg to 1000 mg, or equivalent, of oral or intravenous acetaminophen.

Embodiment 15. The approved product of any one of the previous embodiments, wherein, following administration, the median duration of response is 9 months.

Embodiment 16. A combination of a corticosteroid, a histamine-1 (H1) receptor antagonist, an antipyretic, and an approved product.

Embodiment 17. The combination of embodiment 16, wherein the corticosteroid comprises oral or intravenous dexamethasone, the H1 receptor antagonist comprises oral or intravenous diphenhydramine, and the antipyretic comprises oral or intravenous acetaminophen.

Embodiment 18. The combination of embodiment 17, wherein the combination comprises 16 mg of oral or intravenous dexamethasone, 50 mg or equivalent of oral or intravenous diphenhydramine, and 650 mg to 1000 mg, or equivalent, of oral or intravenous acetaminophen.

Embodiment 19. A pharmaceutical product comprising teclistamab-cqyv in a single-dose vial, wherein the pharmaceutical product is packaged, and wherein the package includes a label that identifies the pharmaceutical product as indicated as monotherapy for the treatment of adult patients with relapsed or refractory multiple myeloma, who have received at least four prior therapies, including an immunomodulatory agent, a proteasome inhibitor, and an anti-CD38 monoclonal antibody.

Embodiment 20. A kit comprising the approved product of any one of embodiments 1-15, the combination of any one of embodiments 16-18, or the pharmaceutical product of embodiment 19.

Embodiment 21. A method for treating relapsed or refractory multiple myeloma in a patient, the method comprising administering the approved product of any one of embodiments 1-15 or the combination of any one of embodiments 16-18 to the patient in an amount and manner that is described in a drug product label for the approved product.

Embodiment 22. The method of embodiment 21, further comprising, prior to the administering of the approved product, administering a corticosteroid, a histamine-1 (H1) receptor antagonist, and an antipyretic to the patient.

Embodiment 23. The method of embodiment 22, wherein the corticosteroid is dexamethasone, the H1 receptor antagonist is diphenhydramine, and the antipyretic is acetaminophen.

Embodiment 24. The method of embodiment 23, comprising administering 16 mg of oral or intravenous dexamethasone, 50 mg or equivalent of oral or intravenous diphenhydramine, and 650 mg to 1000 mg, or equivalent, of oral or intravenous acetaminophen, prior to the administering of the approved product.

Embodiment 25. The method of any one of embodiments 22-24, wherein the corticosteroid, the H1 receptor antagonist, and the antipyretic are administered to the patient prior to a step-up dose or a treatment dose.

Embodiment 26. A method for treating relapsed or refractory multiple myeloma in a patient, the method comprising administering a corticosteroid, a histamine-1 (H1) receptor antagonist, an antipyretic, and the approved product of any one of embodiments 1-15 to the patient in an amount and manner that is described in a drug product label for the approved product.

Embodiment 27. The method of embodiment 26, comprising administering 16 mg of oral or intravenous dexamethasone, 50 mg or equivalent of oral or intravenous diphenhydramine, and 650 mg to 1000 mg, or equivalent, of oral or intravenous acetaminophen, prior to administering the approved product.

Embodiment 28. A method of selling an approved product, said method comprising selling such approved product, wherein an approved product label for a reference product for such approved product includes instructions for treating adult patients with relapsed or refractory multiple myeloma after at least four prior lines of therapy, including a proteasome inhibitor, an immunomodulatory agent, and an anti-CD38 monoclonal antibody.

Embodiment 29. A method of offering for sale an approved product, said method comprising offering for sale such approved product, wherein an approved product label for a reference product for such approved product includes instructions for treating adult patients with relapsed or refractory multiple myeloma after at least four prior lines of therapy, including a proteasome inhibitor, an immunomodulatory agent, and an anti-CD38 monoclonal antibody.

Embodiment 30. A method of negotiating a maximum fair price for an approved product, the method comprising identifying the approved product as negotiation eligible, selecting said negotiation eligible approved product to be included in a price negotiation with a license holder of said approved product, wherein said license holder and a third party enter into negotiations to determine a maximum fair price for said negotiation eligible approved product, and concluding said negotiations with an agreed-upon maximum fair price for said negotiation eligible approved product.

Embodiment 31. The approved product of any of embodiments 1-15, wherein the approved product is administered to a patient in accordance with a REMS program to reduce a risk that the patient experiences cytokine release syndrome (CRS) and/or neurologic toxicity (e.g., ICANS) after receiving the approved product.

Embodiment 32. The pharmaceutical product of embodiment 19, wherein the label includes a warning that cytokine release syndrome (CRS) and/or neurologic toxicity (e.g., ICANS) can occur in patients that receive the pharmaceutical product.

Embodiment 33. The pharmaceutical product of embodiment 19 or 32, wherein the label indicates that the pharmaceutical product is available only through a REMS program.

Embodiment 34. The method of any of embodiments 21-27 comprising reducing the patient's risk of CRS and/or neurologic toxicity (e.g., ICANS) by administering the approved product to the patient in accordance with a REMS program.

Embodiment 35. An approved drug product with at least one approved indication, wherein said approved drug product comprises teclistamab-cqyv.

Embodiment 36. A drug product comprising teclistamab-cqyv, wherein the drug product is approved for treating multiple myeloma.

Embodiment 37. An approved drug product comprising teclistamab-cqyv, wherein such approved drug product improves anti-cancer cell activity as measured by overall response rate, duration of response (DOR), DOR rate, or time to first response, relative to a patient with multiple myeloma who is not receiving treatment with a bispecific antibody.

Embodiment 38. An approved drug product that comprises teclistamab-cqyv and is reference listed on the basis of data demonstrating an improvement in anti-cancer cell activity as measured by overall response rate, duration of response (DOR), DOR rate, or time to first response, relative to a patient with multiple myeloma who is not receiving treatment with a bispecific antibody.

Provided below are additional enumerated embodiments. These embodiments are illustrative only and do not limit the scope of the present disclosure or of the claims attached hereto.

1A. A method of restarting a subject's dosing regimen for a BCMAxCD3 bispecific antibody after the subject has initiated a planned dosing regimen for treatment of multiple myeloma, and a delay has occurred between doses of the BCMAxCD3 bispecific antibody during the planned dosing regimen, wherein the planned dosing regimen comprises:
(1) a step-up dosing schedule that includes (a) a first step-up dose of 0.06 mg/kg, (b) a second step-up dose of 0.3 mg/kg between 2 to 7 days after the first step-up dose, and (c) a first treatment dose of 1.5 mg/kg between 2 to 7 days after the second step-up dose; and
(2) a weekly dosing schedule that includes subsequent treatment doses of 1.5 mg/kg one week after the first treatment dose and once weekly thereafter,
wherein the method of restarting the subject's dosing regimen comprises:
(i) if the last dose administered to the subject was the first step-up dose of 0.06 mg/kg, and the time from the last dose administered is more than 7 days, then restarting the step-up dosing schedule at the first step-up dose of 0.06 mg/kg and resuming the planned dosing regimen, or
(ii) if the last dose administered to the subject was the second step-up dose of 0.3 mg/kg, and the time from the last dose administered is 8 to 28 days, then repeating the second step-up dose of 0.3 mg/kg and continuing the step-up dosing schedule (thereby resuming the planned dosing regimen), or
(iii) if the last dose administered to the subject was the second step-up dose of 0.3 mg/kg, and the time from the last dose administered is more than 28 days, then restarting the step-up dosing schedule at the first step-up dose of 0.06 mg/kg and resuming the planned dosing regimen, or
(iv) if the last dose administered to the subject was 1.5 mg/kg, and the time from the last dose administered is 8 to 28 days, then continuing the planned dosing regimen at treatment doses of 1.5 mg/kg weekly (thereby resuming the planned dosing regimen), or
(v) if the last dose administered to the subject was 1.5 mg/kg, and the time from the last dose administered is more than 28 days, then restarting the step-up dosing schedule at the first step-up dose of 0.06 mg/kg and resuming the planned dosing regimen.
   2A. A method of restarting a subject's dosing regimen for a BCMAxCD3 bispecific antibody after the subject has initiated a planned dosing regimen for treatment of multiple myeloma, and a delay has occurred between doses of the BCMAxCD3 bispecific antibody during the planned dosing regimen, wherein the planned dosing regimen comprises:
(1) a step-up dosing schedule that includes (a) a first step-up dose of 0.06 mg/kg, (b) a second step-up dose of 0.3 mg/kg between 2 to 7 days after the first step-up dose, and (c) a first treatment dose of 1.5 mg/kg between 2 to 7 days after the second step-up dose; and
(2) a weekly dosing schedule that includes subsequent treatment doses of 1.5 mg/kg one week after the first treatment dose and once weekly thereafter; and
(3) if the subject has any response for a minimum of 6 months, a bi-weekly dosing schedule that includes subsequent treatment doses of 1.5 mg/kg once every two weeks,
wherein the method of restarting the subject's dosing regimen comprises:
(i) if the last dose administered to the subject was the first step-up dose of 0.06 mg/kg, and the time from the last dose administered is more than 7 days, then restarting the step-up dosing schedule at the first step-up dose of 0.06 mg/kg and resuming the planned dosing regimen, or
(ii) if the last dose administered to the subject was the second step-up dose of 0.3 mg/kg, and the time from the last dose administered is 8 to 28 days, then repeating the second step-up dose of 0.3 mg/kg and continuing the step-up dosing schedule (thereby resuming the planned dosing regimen), or
(iii) if the last dose administered to the subject was the second step-up dose of 0.3 mg/kg, and the time from the last dose administered is more than 28 days, then restarting the step-up dosing schedule at the first step-up dose of 0.06 mg/kg and resuming the planned dosing regimen, or
(iv) if the last dose administered to the subject was 1.5 mg/kg, and the time from the last dose administered is 8 to 28 days, then continuing the planned dosing regimen at the last treatment dose and schedule (treatment doses of 1.5 mg/kg weekly or 1.5 mg/kg bi-weekly), or
(v) if the last dose administered to the subject was 1.5 mg/kg, and the time from the last dose administered is more than 28 days, then restarting the step-up dosing schedule at the first step-up dose of 0.06 mg/kg and resuming the planned dosing regimen.
   3A. A method of restarting a subject's dosing regimen for a BCMAxCD3 bispecific antibody after the subject has initiated a planned dosing regimen for treatment of multiple myeloma, and a delay has occurred between doses of the BCMAxCD3 bispecific antibody during the planned dosing regimen, wherein the planned dosing regimen comprises:
(1) a step-up dosing schedule that includes (a) a first step-up dose of 0.06 mg/kg, (b) a second step-up dose of 0.3 mg/kg between 2 to 7 days after the first step-up dose, and (c) a first treatment dose of 1.5 mg/kg between 2 to 7 days after the second step-up dose; and
(2) a weekly dosing schedule that includes subsequent treatment doses of 1.5 mg/kg one week after the first treatment dose and once weekly thereafter; and
(3) if the subject has a complete response or better for a minimum of 6 months, a bi-weekly dosing schedule that includes subsequent treatment doses of 1.5 mg/kg once every two weeks,
wherein the method of restarting the subject's dosing regimen comprises:
(i) if the last dose administered to the subject was the first step-up dose of 0.06 mg/kg, and the time from the last dose administered is more than 7 days, then restarting the step-up dosing schedule at the first step-up dose of 0.06 mg/kg and resuming the planned dosing regimen, or
(ii) if the last dose administered to the subject was the second step-up dose of 0.3 mg/kg, and the time from the last dose administered is 8 to 28 days, then repeating the second step-up dose of 0.3 mg/kg and continuing the step-up dosing schedule (thereby resuming the planned dosing regimen), or
(iii) if the last dose administered to the subject was the second step-up dose of 0.3 mg/kg, and the time from the last dose administered is more than 28 days, then restarting the step-up dosing schedule at the first step-up dose of 0.06 mg/kg and resuming the planned dosing regimen, or
(iv) if the last dose administered to the subject was 1.5 mg/kg, and the time from the last dose administered is 8 to 28 days, then continuing the planned dosing regimen at the last treatment dose and schedule (treatment doses of 1.5 mg/kg weekly or 1.5 mg/kg bi-weekly), or
(v) if the last dose administered to the subject was 1.5 mg/kg, and the time from the last dose administered is more than 28 days, then restarting the step-up dosing schedule at the first step-up dose of 0.06 mg/kg and resuming the planned dosing regimen.
   4A. The method of any of embodiments 1A-3A, wherein the step-up dosing schedule includes (a) the first step-up dose of 0.06 mg/kg on Day 1, (b) the second step-up dose of 0.3 mg/kg on Day 4, and (c) the first treatment dose of 1.5 mg/kg on Day 7.
   5A. The method of any of embodiments 1A-3A, wherein each of the second step-up dose and the first treatment dose is administered between 2 to 4 days after the previous dose, or is optionally administered up to 7 days after the previous dose to allow for resolution of adverse reactions.
   6A. The method of any of embodiments 1A-5A further comprising administering pretreatment medications to the subject prior to restarting the planned dosing regimen, wherein the pretreatment medications comprise 16 mg or equivalent of oral or intravenous dexamethasone, 50 mg or equivalent of oral or intravenous diphenhydramine, and 650 mg to 1000 mg, or equivalent, oral or intravenous acetaminophen.
   7A. The method of any of embodiments 1A-6A, wherein the BCMAxCD3 bispecific antibody comprises a BCMA binding domain comprising the HCDR1 of SEQ ID NO: 5, the HCDR2 of SEQ ID NO: 6, the HCDR3 of SEQ ID NO: 7, the LCDR1 of SEQ ID NO: 8, the LCDR2 of SEQ ID NO: 9 and the LCDR3 of SEQ ID NO: 10, and a CD3 binding domain comprising the HCDR1 of SEQ ID NO: 11, the HCDR2 of SEQ ID NO: 12, the HCDR3 of SEQ ID NO: 13, the LCDR1 of SEQ ID NO: 14, the LCDR2 of SEQ ID NO: 15 and the LCDR3 of SEQ ID NO: 16.
   8A. The method of any of embodiments 1A-7A, wherein the BCMAxCD3 bispecific antibody is an IgG1, an IgG2, an IgG3 or an IgG4 isotype.
   9A. The method of any of embodiments 1A-8A, wherein the BCMAxCD3 bispecific antibody is an IgG4 isotype.
   10A. The method of any of embodiments 1A-9A, wherein the BCMAxCD3 bispecific antibody comprises one or more substitutions in its Fc region.
   11A. The method of any of embodiments 1A-10A, wherein the BCMAxCD3 bispecific antibody is an IgG4 isotype and comprises Proline/Alanine/Alanine substitutions at amino acid positions 228/234/235, respectively, in its Fc region (according to EU index numbering).
   12A. The method of any of embodiments 1A-11A, wherein the BCMAxCD3 bispecific antibody is an IgG4 isotype and comprises F405L and R409K substitutions in its Fc region (according to EU index numbering).
   13A. The method of any of embodiments 1A-12A, wherein the Fc region of the BCMA-binding arm comprises Proline/Alanine/Alanine substitutions at amino acid positions 228/234/235, respectively (according to EU index numbering).
   14A. The method of any of embodiments 1A-13A, wherein the Fc region of the CD3-binding arm comprises Proline/Alanine/Alanine substitutions at amino acid positions 228/234/235, respectively, in addition to F405L and R409K substitutions (according to EU index numbering).
   15A. The method of any of embodiments 1A-14A, wherein the BCMAxCD3 bispecific antibody comprises a first heavy chain (HCl) having the amino acid sequence of SEQ ID NO: 1, a first light chain (LC1) having the amino acid sequence of SEQ ID NO: 2, a second heavy chain (HC2) having the amino acid sequence of SEQ ID NO: 3 and a second light chain (LC2) having the amino acid sequence of SEQ ID NO: 4.
   16A. The method of any of embodiments 1A-14A, wherein the BCMAxCD3 bispecific antibody comprises a first heavy chain (HCl) having at least 90% identity to the amino acid sequence of SEQ ID NO: 1, a first light chain (LC1) having at least 90% identity to the amino acid sequence of SEQ ID NO: 2, a second heavy chain (HC2) having at least 90% identity to the amino acid sequence of SEQ ID NO: 3 and a second light chain (LC2) having at least 90% identity to the amino acid sequence of SEQ ID NO: 4.
   17A. The method of any of embodiments 1A-14A, wherein the BCMAxCD3 bispecific antibody comprises a first heavy chain (HCl) having at least 95% identity to the amino acid sequence of SEQ ID NO: 1, a first light chain (LC1) having at least 95% identity to the amino acid sequence of SEQ ID NO: 2, a second heavy chain (HC2) having at least 95% identity to the amino acid sequence of SEQ ID NO: 3 and a second light chain (LC2) having at least 95% identity to the amino acid sequence of SEQ ID NO: 4.
   18A. The method of any of embodiments 1A-14A, wherein the BCMAxCD3 bispecific antibody comprises a first heavy chain (HCl) having at least 98% identity to the amino acid sequence of SEQ ID NO: 1, a first light chain (LC1) having at least 98% identity to the amino acid sequence of SEQ ID NO: 2, a second heavy chain (HC2) having at least 98% identity to the amino acid sequence of SEQ ID NO: 3 and a second light chain (LC2) having at least 98% identity to the amino acid sequence of SEQ ID NO: 4.
   19A. The method of any of embodiments 1A-14A, wherein the BCMAxCD3 bispecific antibody comprises a first heavy chain (HCl) having at least 99% identity to the amino acid sequence of SEQ ID NO: 1, a first light chain (LC1) having at least 99% identity to the amino acid sequence of SEQ ID NO: 2, a second heavy chain (HC2) having at least 99% identity to the amino acid sequence of SEQ ID NO: 3 and a second light chain (LC2) having at least 99% identity to the amino acid sequence of SEQ ID NO: 4.
   20A. The method of any of embodiments 1A-15A, wherein the BCMAxCD3 bispecific antibody is teclistamab.
   21A. The method of any of embodiments 1A-20A, wherein the subject has relapsed or refractory multiple myeloma.
   22A. The method of any of embodiments 1A-21A, wherein the subject has relapsed and refractory multiple myeloma and has received at least 3 prior therapies, including an immunomodulatory agent, a proteasome inhibitor, and an anti-CD38 antibody and has demonstrated disease progression on the last therapy.
   23A. The method of any of embodiments 1A-21A, wherein the subject has relapsed or refractory multiple myeloma and has received at least four prior lines of therapy, including a proteasome inhibitor, an immunomodulatory agent and an anti-CD38 monoclonal antibody.
   24A. The method of any of embodiments 1A-23A, wherein each dose of 0.06 mg/kg and each dose of 0.3 mg/kg is administered as 10 mg/mL of the bispecific antibody in solution for injection.
   25A. The method of any of embodiments 1A-24A, wherein each dose of 1.5 mg/kg is administered as 90 mg/mL of the bispecific antibody.
   26A. The method of any of embodiments 1A-25A, wherein the BCMAxCD3 bispecific antibody is an approved product.
   27A. The method of embodiment 26A, wherein the approved product is a biosimilar.

The examples and embodiments described herein are for illustrative purposes only and various modifications or changes suggested to persons skilled in the art are to be included within the spirit and purview of this application and scope of the appended claims.

What is claimed:

1. An approved product comprising:
30 mg/3 mL (10 mg/mL) of teclistamab-cqyv in a single-dose vial; or
153 mg/1.7 mL (90 mg/mL) of teclistamab-cqyv in a single-dose vial.

2. The approved product of claim 1, comprising 30 mg/3 mL (10 mg/mL) of teclistamab-cqyv in a single-dose vial.

3. The approved product of claim 1, comprising 153 mg/1.7 mL (90 mg/mL) of teclistamab-cqyv in a single-dose vial.

4. The approved product of claim 1, wherein the approved product is formulated for delivery to a human in a dosing schedule comprising:
0.06 mg/kg administered as a first step-up dose on Day 1;
0.3 mg/kg administered as a second step-up dose 2 to 7 days after the first step-up dose;
1.5 mg/kg administered as a first treatment dose 2 to 7 days after the second step-up dose; and
1.5 mg/kg administered as a subsequent treatment dose one week after the first treatment dose and once weekly thereafter.

5. The approved product of claim 4, wherein the second step-up dose is administered on Day 4.

6. The approved produce of claim 4, wherein the first treatment dose is administered on Day 7.

7. The approved product of claim 1, wherein the approved product is formulated for delivery to a human in a dosing schedule comprising:
0.06 mg/kg administered as a first step-up dose on Day 1;
0.3 mg/kg administered as a second step-up dose on Day 4;
1.5 mg/kg administered as a first treatment dose on Day 7; and
1.5 mg/kg administered as a subsequent treatment dose one week after the first treatment dose and once weekly thereafter.

8. The approved product of claim 1, wherein the approved product is a biosimilar.

9. The approved product of claim 1, in a 3 ml single-dose vial comprising 30 mg of teclistamab-cqyv, edetate disodium, glacial acetic acid, polysorbate 20, sodium acetate, sucrose, and water for injection, USP.

10. The approved product of claim 1, in a 3 ml single-dose vial comprising 30 mg of teclistamab-cqyv, 0.054 mg edetate disodium, 0.72 mg glacial acetic acid, 1.2 mg polysorbate 20, 2.7 mg sodium acetate, 240 mg sucrose, and water for injection, USP.

11. The approved product of claim 1, in a 1.7 ml single-dose vial comprising 153 mg of teclistamab-cqyv, edetate disodium, glacial acetic acid, polysorbate 20, sodium acetate, sucrose, and water for injection, USP.

12. The approved product of claim 1, in a 1.7 ml single-dose vial comprising 153 mg of teclistamab-cqyv, 0.031 mg edetate disodium, 0.41 mg glacial acetic acid, 0.68 mg polysorbate 20, 1.5 mg sodium acetate, 140 mg sucrose, and water for injection, USP.

13. The approved product of claim 1, in combination with a corticosteroid, a histamine-1 (H1) receptor antagonist, and an antipyretic.

14. The approved product of claim 13, in combination with 16 mg of oral or intravenous dexamethasone, 50 mg or equivalent of oral or intravenous diphenhydramine, and 650 mg to 1000 mg, or equivalent, of oral or intravenous acetaminophen.

15. The approved product of claim 1, wherein, following administration, the median duration of response is 9 months.

16. A combination of a corticosteroid, a histamine-1 (H1) receptor antagonist, an antipyretic, and an approved product.

17. The combination of claim 16, wherein the corticosteroid comprises oral or intravenous dexamethasone, the H1 receptor antagonist comprises oral or intravenous diphenhydramine, and the antipyretic comprises oral or intravenous acetaminophen.

18. The combination of claim 17, wherein the combination comprises 16 mg of oral or intravenous dexamethasone, 50 mg or equivalent of oral or intravenous diphenhydramine, and 650 mg to 1000 mg, or equivalent, of oral or intravenous acetaminophen.

19. A pharmaceutical product comprising teclistamab-cqyv in a single-dose vial, wherein the pharmaceutical product is packaged, and wherein the package includes a label that identifies the pharmaceutical product as indicated as monotherapy for the treatment of adult patients with relapsed or refractory multiple myeloma, who have received at least four prior therapies, including an immunomodulatory agent, a proteasome inhibitor, and an anti-CD38 monoclonal antibody.

20. A kit comprising the approved product of claim 1; a combination of a corticosteroid, a histamine-1 (H1) receptor antagonist, an antipyretic, and an approved product; or a pharmaceutical product comprising teclistamab-cqyv in a single-dose vial, wherein the pharmaceutical product is packaged, and wherein the package includes a label that identifies the pharmaceutical product as indicated as monotherapy for the treatment of adult patients with relapsed or refractory multiple myeloma, who have received at least four prior therapies, including an immunomodulatory agent, a proteasome inhibitor, and an anti-CD38 monoclonal antibody.

21. A method for treating relapsed or refractory multiple myeloma in a patient, the method comprising administering the approved product of claim 1, or a combination of a corticosteroid, a histamine-1 (H1) receptor antagonist, an antipyretic, and an approved product, to the patient in an amount and manner that is described in a drug product label for the approved product.

22. The method of claim 21, further comprising, prior to the administering of the approved product, administering a corticosteroid, a histamine-1 (H1) receptor antagonist, and an antipyretic to the patient.

23. The method of claim 22, wherein the corticosteroid is dexamethasone, the H1 receptor antagonist is diphenhydramine, and the antipyretic is acetaminophen.

24. The method of claim 23, comprising administering 16 mg of oral or intravenous dexamethasone, 50 mg or equivalent of oral or intravenous diphenhydramine, and 650 mg to 1000 mg, or equivalent, of oral or intravenous acetaminophen, prior to the administering of the approved product.

25. The method of claim 22, wherein the corticosteroid, the H1 receptor antagonist, and the antipyretic are administered to the patient prior to a step-up dose or a treatment dose.

26. A method for treating relapsed or refractory multiple myeloma in a patient, the method comprising administering a corticosteroid, a histamine-1 (H1) receptor antagonist, an antipyretic, and the approved product of claim 1 to the patient in an amount and manner that is described in a drug product label for the approved product.

27. The method of claim 26, comprising administering 16 mg of oral or intravenous dexamethasone, 50 mg or equivalent of oral or intravenous diphenhydramine, and 650 mg to 1000 mg, or equivalent, of oral or intravenous acetaminophen, prior to administering the approved product.

28. A method of selling an approved product, said method comprising selling such approved product, wherein an approved product label for a reference product for such approved product includes instructions for treating adult patients with relapsed or refractory multiple myeloma after at least four prior lines of therapy, including a proteasome inhibitor, an immunomodulatory agent, and an anti-CD38 monoclonal antibody.

29. A method of offering for sale an approved product, said method comprising offering for sale such approved product, wherein an approved product label for a reference product for such approved product includes instructions for treating adult patients with relapsed or refractory multiple myeloma after at least four prior lines of therapy, including a proteasome inhibitor, an immunomodulatory agent, and an anti-CD38 monoclonal antibody.

30. A method of negotiating a maximum fair price for an approved product, the method comprising identifying the approved product as negotiation eligible, selecting said negotiation eligible approved product to be included in a price negotiation with a license holder of said approved product, wherein said license holder and a third party enter into negotiations to determine a maximum fair price for said negotiation eligible approved product, and concluding said negotiations with an agreed-upon maximum fair price for said negotiation eligible approved product.

31. The approved product of claim 1, wherein the approved product is administered to a patient in accordance with a REMS program to reduce a risk that the patient experiences cytokine release syndrome (CRS) and/or neurologic toxicity (e.g., ICANS) after receiving the approved product.

32. The pharmaceutical product of claim 19, wherein the label includes a warning that cytokine release syndrome (CRS) and/or neurologic toxicity (e.g., ICANS) can occur in patients that receive the pharmaceutical product.

33. The pharmaceutical product of claim 19, wherein the label indicates that the pharmaceutical product is available only through a REMS program.

34. The method of claim 21 comprising reducing the patient's risk of CRS and/or neurologic toxicity (e.g., ICANS) by administering the approved product to the patient in accordance with a REMS program.

35. An approved drug product with at least one approved indication, wherein said approved drug product comprises teclistamab-cqyv.

36. A drug product comprising teclistamab-cqyv, wherein the drug product is approved for treating multiple myeloma.

37. An approved drug product comprising teclistamab-cqyv, wherein such approved drug product improves anti-cancer cell activity as measured by overall response rate, duration of response (DOR), DOR rate, or time to first response, relative to a patient with multiple myeloma who is not receiving treatment with a bispecific antibody.

38. An approved drug product that comprises teclistamab-cqyv and is reference listed on the basis of data demonstrating an improvement in anti-cancer cell activity as measured by overall response rate, duration of response (DOR), DOR rate, or time to first response, relative to a patient with multiple myeloma who is not receiving treatment with a bispecific antibody.


SEQUENCE LISTING (16 sequences)

SEQ ID NO: 1 - protein, 448 aa, synthetic construct
QLQLQESGPGLVKPSETLSLTCTVSGGSISSGSYFWGWIRQPPGKGLEWIGSIYYSGITYYNPSLKSRVTISVDTSKNQFSLKLSSVTAADTAVYYCARHDGAVAGLFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 2 - protein, 214 aa, synthetic construct
SYVLTQPPSVSVAPGQTARITCGGNNIGSKSVHWYQQPPGQAPVVVVYDDSDRPSGIPERFSGSNSGNTATLTISRVEAGDEAVYYCQVWDSSSDHVVFGGGTKLTVLGQPKAAPSVTLFPPSSEELQANKATLVCLISDFYPGAVTVAWKGDSSPVKAGVETTTPSKQSNNKYAASSYLSLTPEQWKSHRSYSCQVTHEGSTVEKTVAPTECS

SEQ ID NO: 3 - protein, 452 aa, synthetic construct
EVQLVESGGGLVQPGGSLRLSCAASGFTFNTYAMNWVRQAPGKGLEWVARIRSKYNNYATYYAASVKGRFTISRDDSKNSLYLQMNSLKTEDTAVYYCARHGNFGNSYVSWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFLLYSKLTVDKSRWQEGNVFSCSVMHEALHNHYTQKSLSLSLGK

SEQ ID NO: 4 - protein, 215 aa, synthetic construct
QTVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 5 - protein, 10 aa, synthetic construct
GGSISSGSYF

SEQ ID NO: 6 - protein, 16 aa, synthetic construct
SIYYSGITYYNPSLKS

SEQ ID NO: 7 - protein, 11 aa, synthetic construct
HDGAVAGLFDY

SEQ ID NO: 8 - protein, 11 aa, synthetic construct
GGNNIGSKSVH

SEQ ID NO: 9 - protein, 7 aa, synthetic construct
DDSDRPS

SEQ ID NO: 10 - protein, 11 aa, synthetic construct
QVWDSSSDHVV

SEQ ID NO: 11 - protein, 10 aa, synthetic construct
GFTFNTYAMN

SEQ ID NO: 12 - protein, 13 aa, synthetic construct
RIRSKYNNYATYY

SEQ ID NO: 13 - protein, 14 aa, synthetic construct
HGNFGNSYVSWFAY

SEQ ID NO: 14 - protein, 14 aa, synthetic construct
RSSTGAVTTSNYAN

SEQ ID NO: 15 - protein, 7 aa, synthetic construct
GTNKRAP

SEQ ID NO: 16 - protein, 9 aa, synthetic construct
ALWYSNLWV